(19) United States
(12) Patent Application Publication
(10) Pub. No.: US 2016/0215061 A1
(43) Pub. Date: Jul. 28, 2016

(54) Fc CONTAINING POLYPEPTIDES HAVING INCREASED BINDING TO FcGammaRIIB

(71) Applicant: MERCK SHARP & DOHME CORP., Rahway, NJ (US)

(72) Inventors: Hussam H. Shaheen, Lebanon, NH (US); Andy Stadheim, Lyme, NH (US); Natarajan Sethuraman, Hanover, NH (US)

(73) Assignee: Merck Sharp & Dohme Corp., Rahway, NJ (US)

(21) Appl. No.: 15/024,918

(22) PCT Filed: Oct. 3, 2014
(86) PCT No.: PCT/US14/58937

§ 371 Date: Mar. 25, 2016

Related U.S. Application Data
(60) Provisional application No. 61/888,009, filed on Oct. 8, 2013.

(51) Int. Cl. C07K 16/32

(52) U.S. Cl. CPC C07K 16/32; C07K 2317/92; C07K 2317/52; C07K 2317/524; C07K 2317/526

(57) ABSTRACT

The present invention is directed to methods and compositions for the production of Fc-containing polypeptides which are useful as human or animal therapeutic agents, and which comprise increased anti-inflammatory properties and improved FcyRIIb binding.

### FIELD OF THE INVENTION

The present invention is directed to methods and compositions for the production of Fc-containing polypeptides which are useful as human or animal therapeutic agents, and which comprise increased binding to FcγRIIB.

### BACKGROUND OF THE INVENTION

Monoclonal antibodies often achieve their therapeutic benefit through two binding events—the binding of the Fab portion to an antigen, and the binding of the Fc portion to an Fc receptor. The binding of an antibody to an Fc receptor (FcR) mediates the “effector function” of the antibody. Because FcRs mediate antibody effector function by binding to the Fc region of the receptor's cognate antibody, FcRs are defined by their specificity for immunoglobulin subtypes. Thus FcRs specific for IgG antibodies are referred to as Fcγ receptors (“FcγRs”).

There are various FcγRs, and in certain cases it is useful to engineer antibodies or antibody fragments that have selectively enhanced binding to certain receptors. Generally, FcγRs can be activating or inhibitory. FcγRIa, FcγRIIa and FcγRIIIa are activating receptors. It has been reported that antibodies that have been engineered to have increased binding to FcγRIIIa exhibit superior ADCC activity and antitimuor activity when compared to their wild-type counterpart.

FcγRIIb is an inhibitory receptor reported to function as an immunomodulatory receptor. The inhibitory FcγRIIb plays a critical role in regulating B-cell homeostasis and regulating other B-cell stimulators that amplify B-cell proliferation and differentiation and suppresses the expression of costimulatory molecules. It has been reported that introducing S267E and L328F mutations in the FcγR of an antibody (wherein the numbering is according to Kabat) can selectively increase the binding affinity to FcγRIIb. However, this mutation has also been reported to retain binding to one of the FcγRIIa allotypes, FcγRIIa^{R131}. See Smith et al., PNAS 109:6181-6186 (2012). It has been suggested that antibodies having that have been engineered to have increased binding to FcγRIIb have beneficial therepautic properties, such as enhanced anti-inflammatory activity. See Mimoto et al., Protein Engineering, Design and Selection, pp 1-10 (Jun. 5, 2013). It has also been reported that antibodies having increased binding to FcγRIIb can increase the cytotoxic activity of immunomodulatory antibodies, such as anti-TNFR antibodies. See White et al., Cancer Immunol. Immunother. 62:941-948 (2013); Li and Ravetch, PNAS 109(27):10966-10971 (2013); Li and Ravetch, Cell Cycle 11:18, 3343-3344 (2012).

Given the importance of monoclonal antibodies as therapeutics, it is desirable to identify methods that can further improve the therapeutic efficacy of these antibodies. It would be desirable to engineer antibodies or other immunotherapeutics which have enhanced binding to FcγRIIb.

### SUMMARY OF THE INVENTION

### Fc-Containing Polypeptides

The invention comprises an Fc-containing polypeptide (such as antibody or an antibody fragment or immunoadhesin) comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat, wherein the polypeptide comprises altered binding to human and mouse FcγRIIB when compared to a parent Fc-containing polypeptide. In one embodiment, the Fc-containing polypeptide comprises a mutation selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P.

The invention comprises an Fc-containing polypeptide (such as antibody or an antibody fragment or immunoadhesin) comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat, wherein the polypeptide comprises enhanced binding to human and mouse FcγRIIB when compared to a parent Fc-containing polypeptide. In one embodiment, the Fc-containing polypeptide comprises a mutation selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P.

In one embodiment, the Fc-containing polypeptide comprises one or more mutations at positions selected from the group consisting of: 246, 307, 317, 326, 332, 339, 360, 409, 414 and 421 of the Fc region of the Fc-containing polypeptide. In one embodiment, the Fc-containing polypeptide comprises one ore more mutations selected from the group consisting of: K246R, K246E, T307A, K317E, K326E, A339T, K360R, K360E, K409R, K414R, and N421D.

In one embodiment, the Fc-containing polypeptide comprises one or more mutations at positions selected from the group consisting of: 317, 326, and 414 of the Fc region of the Fc-containing polypeptide. In one embodiment, the Fc-containing polypeptide comprises one or more mutations selected from the group consisting of: K317E, K326E, and K414R. In one embodiment, the Fc-containing polypeptide comprises mutations at amino acid positions 317, 326, and 414. In one embodiment, the Fc-containing polypeptide comprises mutations K317E, K326E, and K414R.

In one embodiment, the Fc-containing polypeptide comprises mutations K317E/K326E/A339T/Q342R/K414R.

In one embodiment, the Fc-containing polypeptide comprises mutations V284A/V305A/T307A/K360R.

In one embodiment, the Fc-containing polypeptide comprises mutations T250A/E272K/K360E/V422D/T437A.

In one embodiment, the Fc-containing polypeptide comprises mutations T223I/K320R/K322E.

In one embodiment, the Fc-containing polypeptide comprises mutations L309S/K340E/V412A.

In one embodiment, the Fc-containing polypeptide comprises mutations V302A/K326E/L328M/T335A/Y391H/K409R.

In one embodiment, the Fc-containing polypeptide comprises mutations K246R/A339T/K409R.

In one embodiment, the Fc-containing polypeptide comprises mutations K246E/T307A/K326E.

In one embodiment, the Fc-containing polypeptide comprises mutations N315S/I332T/N421D/S440P.

In one embodiment, the Fc-containing polypeptide comprises mutations K326E/N421D/K439E.

In one embodiment, the Fc-containing polypeptide comprises mutations K326E/I332T/I377V.

In some embodiments, the Fc-containing polypeptides of the invention can further comprise one or more mutations at positions selected from the group consisting of: 241, 243, 252, 254, 256, 264, 267, 328, 339, 342, 433, and 434 of the Fc region, wherein the numbering is according to the EU index as in Kabat. In one embodiment, the Fc-containing polypeptide of the invention comprises mutations at positions 241, 317, 326 and 414, wherein the numbering is according to the EU index as in Kabat. In one embodiment, the Fc-containing polypeptide comprises mutations F241A, K317E, K326E and K414R.

In some embodiments, the Fc-containing polypeptides of the invention comprise N-glycans. In one embodiment, the Fc-containing polypeptides of the invention comprise N-glycans, wherein at least 30%, 40%, 50%, 60%, 70%, 80% or 90% of the N-glycans on the Fc-containing polypeptide comprise an oligosaccharide structure selected from the group consisting of SA(1-4)Gal(1-4)GlcNAc(2-4)Man3GlcNAc2. In one embodiment, the sialic acid residues in the sialylated N-glycans are attached via α-2,6 linkages.

In some embodiments, the Fc-containing polypeptides of the invention are antibodies or antibody fragments. In particular embodiments, the Fc-containing polypeptides of the invention are IgG1 antibodies or antibody fragments. In particular embodiments, the Fc-containing polypeptides of the invention are human IgG1 antibodies or antibody fragments.

In some embodiments, the Fc-containing polypeptides of the invention have one or more of the following properties when compared to a parent Fc-containing polypeptide: reduced effector properties and increase anti-inflammatory property.

### Methods of Producing Fc-Containing Polypeptides

The invention also comprises a method for producing an Fc-containing polypeptide in a host cell comprising: (a) providing a genetically modified host cell that has been genetically engineered to produce an Fc-containing polypeptide comprising sialylated N-glycans, wherein the host cell comprises a nucleic acid encoding the Fc-containing polypeptide comprising one or more mutations at positions selected from the group consisting of 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat; (b) culturing the host cell under conditions which cause expression of the Fc-containing polypeptide; and (c) isolating the Fc-containing polypeptide from the host cell. In one embodiment, the host cell comprises a nucleic acid encoding an Fc-containing polypeptide comprising one or more mutations selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P. In one embodiment, the host cell comprises a nucleic acid encoding an Fc-containing polypeptide comprising mutations K317E/K326E/A339T/Q342R/K414R. In one embodiment, the host cell comprises a nucleic acid encoding an Fc-containing polypeptide comprising mutations V284A/V305A/T307A/K360R. In one embodiment, the host cell comprises a nucleic acid encoding an Fc-containing polypeptide comprising mutations T250A/E272K/K360E/V422D/T437A. In one embodiment, the host cell comprises a nucleic acid encoding an Fc-containing polypeptide comprising mutations T223I/K320R/K322E. In one embodiment, the host cell comprises a nucleic acid encoding an Fc-containing polypeptide comprising mutations L309S/K340E/V412A. In one embodiment, the host cell comprises a nucleic acid encoding an Fc-containing polypeptide comprising mutations V302A/K326E/L328M/T335A/Y391H/K409R. In one embodiment, the host cell comprises a nucleic acid encoding an Fc-containing polypeptide comprising mutations K246R/A339T/K409R. In one embodiment, the host cell comprises a nucleic acid encoding an Fc-containing polypeptide comprising mutations K246E/T307A/K326E. In one embodiment, the host cell comprises a nucleic acid encoding an Fc-containing polypeptide comprising mutations N315S/I332T/N421D/S440P. In one embodiment, the host cell comprises a nucleic acid encoding an Fc-containing polypeptide comprising mutations K326E/N421D/K439E. In one embodiment, the host cell comprises a nucleic acid encoding an Fc-containing polypeptide comprising mutations K326E/I332T/I377V.

The invention also comprises a method for producing an Fc-containing polypeptide in a host cell comprising: (a) providing a genetically modified host cell that has been genetically engineered to produce an Fc-containing polypeptide comprising sialylated N-glycans, wherein the host cell comprises a nucleic acid encoding an Fc-containing polypeptide comprising one or more mutations at positions selected from the group consisting of: 246, 307, 317, 326, 332, 339, 360, 409, 414 and 421 of the Fc region of the Fc-containing polypeptide, wherein the numbering is according to the EU index as in Kabat; (b) culturing the host cell under conditions which cause expression of the Fc-containing polypeptide; and (c) isolating the Fc-containing polypeptide from the host cell. In one embodiment, the host cell comprises a nucleic acid encoding an Fc-containing polypeptide comprising one or more mutations selected from the group consisting of: K246R, K246E, T307A, K317E, K326E, A339T, K360R, K360E, K409R, K414R, and N421D.

The invention also comprises a method for producing a Fc-containing polypeptide in a host cell comprising: (a) providing a genetically modified host cell that has been genetically engineered to produce an Fc-containing polypeptide comprising sialylated N-glycans, wherein the host cell comprises a nucleic acid encoding the Fc-containing polypeptide comprising one or more mutations at positions selected from the group consisting of: 317, 326, and 414 of the Fc region of the Fc-containing polypeptide, wherein the numbering is according to the EU index as in Kabat; (b) culturing the host cell under conditions which cause expression of the Fc-containing polypeptide; and (c) isolating the Fc-containing polypeptide from the host cell. In one embodiment, the host cell comprises a nucleic acid encoding an Fc-containing polypeptide comprising one or more mutation selected from the group consisting of: K317E, K326E, and K414R. In one embodiment, the host cell comprises a nucleic acid encoding an Fc-containing polypeptide comprising mutations K317E, K326E, and K414R.

In some embodiments, the host cells used in the methods of the invention are able to produce Fc-containing polypeptides (such as antibody or antibody fragments) comprising N-glycans. In some embodiments, the host cells used in the methods of the invention can produce Fc-containing polypeptides comprising N-glycans wherein at least 30%, 40%, 50%, 60%, 70%, 80% or 90% of the N-glycans on the Fc-containing polypeptide comprise an oligosaccharide structure selected from the group consisting of SA(1-4)Gal(1-4)GlcNAc(2-4)Man3GlcNAc2. In one embodiment, the sialic acid residues in the sialylated N-glycans are attached via α-2,6 linkages.

### Methods of Increasing the Anti-Inflammatory Properties of an Fc-Containing Polyeptide

The invention also provides a method of increasing the anti-inflammatory properties of an Fc-containing polypeptide comprising introducing one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat; and wherein the Fc-containing polypeptide has improved binding to human and mouse FcγRIIB and increased anti-inflammatory properties when compared to a parent Fc-containing polypeptide. In one embodiment, the invention comprises introducing one or more mutations selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P. In one embodiment, the invention comprises introducing the following mutations: K317E/K326E/A339T/Q342R/K414R. In one embodiment, the invention comprises introducing the following mutations: V284A/V305A/T307A/K360R. In one embodiment, the invention comprises introducing the following mutations: T250A/E272K/K360E/V422D/T437A. In one embodiment, the invention comprises introducing the following mutations: T223I/K320R/K322E. In one embodiment, the invention comprises introducing the following mutations: L309S/K340E/V412A. In one embodiment, the invention comprises introducing the following mutations: V302A/K326E/L328M/T335A/Y391H/K409R. In one embodiment, the invention comprises introducing the following mutations: K246R/A339T/K409R. In one embodiment, the invention comprises introducing the following mutations: K246E/T307A/K326E. In one embodiment, the invention comprises introducing the following mutations: N315S/I332T/N421D/S440P. In one embodiment, the invention comprises introducing the following mutations: K326E/N421D/K439E. In one embodiment, the invention comprises introducing the following mutations: K326E/I332T/I377V.

The invention also provides a method of increasing the anti-inflammatory properties of an Fc-containing polypeptide comprising introducing one or more mutations at positions selected from the group consisting of: 246, 307, 317, 326, 332, 339, 360, 409, 414 and 421 of the Fc region of the Fc-containing polypeptide, wherein the numbering is according to the EU index as in Kabat; and wherein the Fc-containing polypeptide has improved binding to human and mouse FcγRIIB and increased anti-inflammatory properties when compared to a parent Fc-containing polypeptide. In one embodiment, the invention comprises introducing one or more mutations selected from the group consisting of: K246R, K246E, T307A, K317E, K326E, A339T, K360R, K360E, K409R, K414R, and N421D.

The invention also provides a method of increasing the anti-inflammatory properties of an Fc-containing polypeptide comprising introducing one or more mutations at positions selected from the group consisting of: 317, 326, and 414 of the Fc region of the Fc-containing polypeptide, wherein the numbering is according to the EU index as in Kabat; and wherein the Fc-containing polypeptide has improved binding to human and mouse FcγRIIB and increased anti-inflammatory properties when compared to a parent Fc-containing polypeptide. In one embodiment, the invention comprises introducing one or more mutations selected from the group consisting of: K317E, K326E, and K414R. In one embodiment, the invention comprises introducing the mutations: K317E, K326E, and K414R.

In one embodiment, the Fc-containing polypeptide is an antibody or an antibody fragment. In one embodiment, the Fc-containing polypeptide is an IgG1 antibody or an antibody fragment. In one embodiment, the Fc-containing polypeptide is a human IgG1 antibody or an antibody fragment. In one embodiment, the Fc-containing polypeptide is an antibody or an antibody fragment comprising N-glycans.

In some embodiments, the Fc-containing polypeptides comprise N-glycans. In one embodiment, the Fc-containing polypeptides comprise N-glycans, wherein at least 30%, 40%, 50%, 60%, 70%, 80% or 90% of the N-glycans on the Fc-containing polypeptide comprise an oligosaccharide structure selected from the group consisting of SA(1-4)Gal(1-4)GlcNAc(2-4)Man3GlcNAc2. In one embodiment, the sialic acid residues in the sialylated N-glycans are attached via α-2,6 linkages.

### Methods of Treatment

The invention further comprises a method of treating a subject in need thereof comprising: administering to the subject a therapeutically effective amount of an Fc-containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat.

In one embodiment, the subject has an inflammatory condition. In one embodiment, the method further comprises the administration of another anti-inflammatory compound. In another embodiment, the method further comprises the administration of an agent that increases expression of FcγRIIB. In another embodiment, the method further comprises the administration of an agent that binds DC-SIGN.

In one embodiment, the Fc-containing polypeptide administered in this claimed method of treatment comprises one or more mutations at positions selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P. In one embodiment, the Fc-containing polypeptide administered in this claimed method of treatment comprises mutations K317E/K326E/A339T/Q342R/K414R. In one embodiment, the Fc-containing polypeptide administered in this claimed method of treatment comprises mutations V284A/V305A/T307A/K360R. In one embodiment, the Fc-containing polypeptide administered in this claimed method of treatment comprises mutations T250A/E272K/K360E/V422D/T437A. In one embodiment, the Fc-containing polypeptide administered in this claimed method of treatment comprises mutations T223I/K320R/K322E. In one embodiment, the Fc-containing polypeptide administered in this claimed method of treatment comprises mutations L309S/K340E/V412A. In one embodiment, the Fc-containing polypeptide administered in this claimed method of treatment comprises mutations V302A/K326E/L328M/T335A/Y391H/K409R. In one embodiment, the Fc-containing polypeptide administered in this claimed method of treatment comprises mutations K246R/A339T/K409R. In one embodiment, the Fc-containing polypeptide administered in this claimed method of treatment comprises mutations K246E/T307A/K326E. In one embodiment, the Fc-containing polypeptide administered in this claimed method of treatment comprises mutations N315S/I332T/N421D/S440P. In one embodiment, the Fc-containing polypeptide administered in this claimed method of treatment comprises mutations K326E/N421D/K439E. In one embodiment, the Fc-containing polypeptide administered in this claimed method of treatment comprises mutations K326E/I332T/I377V.

In one embodiment, the invention comprises a method of treating a subject in need thereof comprising: administering to the subject a therapeutically effective amount of an Fc-containing polypeptide comprising mutations at one or more positions selected from the group consisting of: 246, 307, 317, 326, 332, 339, 360, 409, 414 and 421 of the Fc region of the Fc-containing polypeptide, wherein the numbering is according to the EU index as in Kabat. In one embodiment, the Fc-containing polypeptide comprises one or more mutations selected from the group consisting of: K246R, K246E, T307A, K317E, K326E, A339T, K360R, K360E, K409R, K414R, and N421D.

In one embodiment, the invention comprises a method of treating a subject in need thereof comprising: administering to the subject a therapeutically effective amount of an Fc-containing polypeptide comprising one or more mutations at positions selected from the group consisting of: 317, 326, and 414 of the Fc region of the Fc-containing polypeptide, wherein the numbering is according to the EU index as in Kabat. In one embodiment, the Fc-containing polypeptide comprises one or more mutation selected from the group consisting of: K317E, K326E, and K414R. In one embodiment, the Fc-containing polypeptide comprises mutations at amino acid positions 317, 326, and 414. In one embodiment, the Fc-containing polypeptide comprises mutations K317E, K326E, and K414R.

The Fc-containing polypeptides to be administered in the claimed methods of treatment can further comprise one or more mutations at positions selected from the group consisting of: 241, 243, 252, 254, 256, 264, 267, 328, 339, 342, 433, and 434 of the Fc region of the Fc-containing polypeptide, wherein the numbering is according to the EU index as in Kabat. In one embodiment, the Fc-containing polypeptide of the invention comprises mutations at positions 241, 317, 326 and 414, wherein the numbering is according to the EU index as in Kabat. In one embodiment, the Fc-containing polypeptide comprises mutations F241A, K317E, K326E and K414R.

The Fc-containing polypeptides to be administered in the claimed methods of treatment can comprise N-glycans. In one embodiment, the Fc-containing polypeptides comprise N-glycans, wherein at least 30%, 40%, 50%, 60%, 70%, 80% or 90% of the N-glycans on the Fc-containing polypeptide comprise an oligosaccharide structure selected from the group consisting of SA(1-4)Gal(1-4)GlcNAc(2-4)Man3GlcNAc2. In one embodiment, the sialic acid residues in the sialylated N-glycans are attached via α-2,6 linkages.

In some embodiments, the Fc-containing polypeptides to be used in the claimed methods of treatment are antibodies or antibody fragments. In particular embodiments, the Fc-containing polypeptides are IgG1 antibodies or antibody fragments. In particular embodiments, the Fc-containing polypeptides are human IgG1 antibodies or antibody fragments.

### Pharmaceutical Compositions

The invention also comprises a pharmaceutical composition comprising: (a) an Fc containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region of the Fc-containing polypeptide, wherein the numbering is according to the EU index as in Kabat, and (b) a pharmaceutically acceptable carrier. In one embodiment, the Fc-containing polypeptide comprises one or more mutations selected from the group consisting of: T223I, K246R, K246E, T250A, E272K, V284A, V305A, V302A, T307A, L309S, K317E, N315S, K320R, K322E, K326E, L328M, I332T, T335A, A339T, K340E, Q342R, K360R, K360E, I377V, Y391H, K409R, V412A, K414R, N421D, V422D, T437A, K439E and S440P. In one embodiment, the Fc-containing polypeptide comprises mutations K317E/K326E/A339T/Q342R/K414R. In one embodiment, the Fc-containing polypeptide comprises mutations V284A/V305A/T307A/K360R. In one embodiment, the Fc-containing polypeptide comprises mutations T250A/E272K/K360E/V422D/T437A. In one embodiment, the Fc-containing polypeptide comprises mutations T223I/K320R/K322E. In one embodiment, the Fc-containing polypeptide comprises mutations L309S/K340E/V412A. In one embodiment, the Fc-containing polypeptide comprises mutations V302A/K326E/L328M/T335A/Y391H/K409R. In one embodiment, the Fc-containing polypeptide comprises mutations K246R/A339T/K409R. In one embodiment, the Fc-containing polypeptide comprises mutations K246E/T307A/K326E. In one embodiment, the Fc-containing polypeptide comprises mutations N315S/I332T/N421D/S440P. In one embodiment, the Fc-containing polypeptide comprises mutations K326E/N421D/K439E. In one embodiment, the Fc-containing polypeptide comprises mutations K326E/I332T/I377V.

The invention also comprises a pharmaceutical composition comprising: (a) an Fc containing polypeptide comprising one or more mutations at positions selected from the group consisting of: 246, 307, 317, 326, 332, 339, 360, 409, 414 and 421 of the Fc region of the Fc-containing polypeptide, wherein the numbering is according to the EU index as in Kabat, and (b) a pharmaceutically acceptable carrier. In one embodiment, the Fc-containing polypeptide comprises one or more mutations selected from the group consisting of: K246R, K246E, T307A, K317E, K326E, A339T, K360R, K360E, K409R, K414R, and N421D.

The invention also comprises a pharmaceutical composition comprising: (a) an Fc containing polypeptide comprising one or more mutations at positions selected from the group consisting of: 317, 326, and 414 of the Fc region of the Fc-containing polypeptide, wherein the numbering is according to the EU index as in Kabat, and (b) a pharmaceutically acceptable carrier. In one embodiment, the Fc-containing polypeptide comprises one or more mutations at positions selected from the group consisting of: K317E, K326E, and K414R. In one embodiment, the Fc-containing polypeptide comprises mutations at amino acid positions 317, 326, and 414. In one embodiment, the Fc-containing polypeptide comprises mutations K317E, K326E, and K414R.

In some embodiments, the Fc-containing polypeptides in the claimed pharmaceutical compositions comprise N-glycans. In one embodiment, the Fc-containing polypeptides comprise N-glycans, wherein at least 30%, 40%, 50%, 60%, 70%, 80% or 90% of the N-glycans on the Fc-containing polypeptide comprise an oligosaccharide structure selected from the group consisting of SA(1-4)Gal(1-4)GlcNAc(2-4)Man3GlcNAc2. In one embodiment, the sialic acid residues in the sialylated N-glycans are attached via α-2,6 linkages.

In some embodiments, the Fc-containing polypeptides in the claimed pharmaceutical compositions are antibodies or antibody fragments. In particular embodiments, the Fc-containing polypeptides are IgG1 antibodies or antibody fragments. In particular embodiments, the Fc-containing polypeptides are human IgG1 antibodies or antibody fragments.

### BRIEF DESCRIPTION OF THE DRAWINGS

FIG. 1 illustrates the plasmid designated pGLY11714 containing anti-PCSK9 Lc bait cassette.

FIG. 2 is a schematic representation of the Lc-Sed1p antibody display system described in Example 1. The DNA sequence comprising the IgG light chain (Lc) is fused through a flexible linker to Sed1p. When co-expressed in the same host with a secretable full length IgG molecule, the Lc portion of the anchored fusion (the bait) heterodimerizes in the ER with the heavy chain (Hc) region of the IgG molecule, forming disulfide bridges. Since the surface displayed half IgG molecule can still pair with secreted heavy and light chains (H+L), this complex results in surface display of the full length IgG molecule (two heavy chains paired with two light chains, i.e., H2+L2). Meanwhile the assembly of soluble full length IgG occurs with equal probability resulting in secretion of the bivalent (H2+L2) in the culture medium.

FIG. 3 illustrates the plasmid designated pGLY11576.

FIG. 4 illustrates the plasmid designated pGLY4464.

### DETAILED DESCRIPTION OF THE INVENTION

### Definitions

The term “antibody”, when used herein refers to an immunoglobulin molecule capable of binding to a specific antigen through at least one antigen recognition site located in the variable region of the immunoglobulin molecule. As used herein, the term encompasses not only intact polyclonal or monoclonal antibodies, consisting of four polypeptide chains, i.e. two identical pairs of polypeptide chains, each pair having one “light” chain (LC) (about 25 kDa) and one “heavy” chain (HC) (about 50-70 kDa), but also fragments thereof, such as Fab, Fab′, F(ab′)₂, Fv, single chain (ScFv), mutants thereof, fusion proteins comprising an antibody portion, and any other modified configuration of an immunoglobulin molecule that comprises an antigen recognition site and at least the portion of the C_{H}2 domain of the heavy chain immunoglobulin constant region which comprises an N-linked glycosylation site of the C_{H}2 domain, or a variant thereof. As used herein the term includes an antibody of any class, such as IgG (for example, IgG1, IgG2, IgG3 or IgG4), IgM, IgA, IgD and IgE, respectively.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The Fc region of an immunoglobulin comprises two constant domains, CH2 and CH3, and can optionally comprise a hinge region. In one embodiment, the Fc region comprises the amino acid sequence of SEQ ID NO: 1 (or a variant thereof comprising point mutations). In one embodiment, the Fc region comprises the amino acid sequence of SEQ ID NO:2 (or a variant thereof comprising point mutations). In another embodiment, the Fc region comprises the amino acid sequence of SEQ ID NO:1 or SEQ ID NO:2 with the addition of a lysine (K) residue at the 3′ end. The Fc region contains a single N-linked glycosylation site in the CH2 domain that corresponds to the Asn297 site of a full-length heavy chain of an antibody, wherein the numbering is according to the EU index as in Kaat.

The term “Fc-containing polypeptide” refers to a polypeptide, such as an antibody or immunoadhesin, which comprises an Fc region or fragment of an Fc region which retains the N-linked glycosylation site in the CH2 domain and retains the ability to recruit immune cells. This term encompasses polypeptides comprising or consisting of (or consisting essentially of) an Fc region either as a monomore or dimeric species. Polypeptides comprising an Fc region can be generated by papain digestion of antibodies or by recombinant DNA technology.

The term “parent antibody”, “parent immunoglobulin” or “parent Fc-containing polypeptide” when used herein refers to an antibody or Fc-containing polypeptide which lacks the Fc region mutations disclosed herein. A parent Fc-containing polypeptide may comprise a native sequence Fc region or an Fc region with pre-existing amino acid sequence modifications. A native sequence Fc region comprises an amino acid sequence identical to the amino acid sequence of an Fc region found in nature. Native sequence Fc regions include the native sequence human IgG1 Fc region, the native sequence human IgG2 Fc region, the native sequence human IgG3 Fc region and the native sequence human IgG4 Fc region as well as naturally occurring variants thereof. When used as a comparator, a parent antibody or a parent Fc-containing polypeptide can be expressed in any cell. In one embodiment, the parent antibody or a parent Fc-containing polypeptide is expressed in the same cell as the Fc-containing polypeptide of the invention.

As used herein, the term “immunoadhesin” designates antibody-like molecules which combine the “binding domain” of a heterologous “adhesin” protein (e.g. a receptor, ligand or enzyme) with an immunoglobulin constant domain. Structurally, the immunoadhesins comprise a fusion of the adhesin amino acid sequence with the desired binding specificity which is other than the antigen recognition and binding site (antigen combining site) of an antibody (i.e. is “heterologous”) and an immunoglobulin constant domain sequence. The term “ligand binding domain” as used herein refers to any native cell-surface receptor or any region or derivative thereof retaining at least a qualitative ligand binding ability of a corresponding native receptor. In a specific embodiment, the receptor is from a cell-surface polypeptide having an extracellular domain that is homologous to a member of the immunoglobulin supergenefamily. Other receptors, which are not members of the immunoglobulin supergenefamily but are nonetheless specifically covered by this definition, are receptors for cytokines, and in particular receptors with tyrosine kinase activity (receptor tyrosine kinases), members of the hematopoietin and nerve growth factor which predispose the mammal to the disorder in question. In one embodiment, the disorder is cancer. Methods of making immunoadhesins are well known in the art. See, e.g., WO00/42072.

The term “Fc mutein” or “Fc mutein antibody” when used herein refers to an Fc-containing polypeptide in which one or more point mutations have been made to the Fc region.

The term “Fc mutation” when used herein refers to a mutation made to the Fc region of an Fc-containing polypeptide.

Throughout the present specification and claims, the numbering of the residues in an immunoglobulin heavy chain or an Fc-containing polypeptide is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

The term “effector function” as used herein refers to a biochemical event that results from the interaction of an antibody Fc region with an Fc receptor or ligand. Exemplary “effector functions” include Clq binding; complement dependent cytotoxicity (CDC); Fc receptor binding; antibody-dependent cell-mediated cytotoxicity (ADCC); phagocytosis; down regulation of cell surface receptors (e. g. B cell receptor; BCR), etc. Such effector functions can be assessed using various assays known in the art.

The terms “Fc receptor” or “FcR” refer to a receptor that binds to the Fc region of an antibody. The preferred Fc receptor is a native sequence of human FcR. Moreover, a preferred FcR is one that binds an IgG antibody—an FcγR—and includes receptors FcγRI, FcγRII, and FcγRIII subclasses, including allelic variants and alternatively spliced forms of these receptors. The term also includes the neonatal receptor, FcRn, that is responsible for the transfer of maternal IgGs to the fetus.

The term FcγRIIB refers to a polyeptide en coded by the human FcRIIB gene, and includes, but is not limited to FcγRIIB1 (GenBank Accession No. NP_003992), FcγRIIB2 (GenBank Accession No. NP_001002273), FcγRIIB3 GenBank Accession No. (NP_001002274), and allelic variants thereof.

The term “glycoengineered Pichia pastoris” when used herein refers to a strain of Pichia pastoris that has been genetically altered to express human-like N-glycans. For example, the GFI 5.0, GFI 5.5 and GFI 6.0 strains.

The term “GFI 5.0” when used herein refers to glycoengineered Pichia pastoris strains that produce glycoproteins having predominantly Gal₂GlcNAc₂Man₃GlcNAc₂ N-glycans.

The term “GFI 6.0” when used herein refers to glycoengineered Pichia pastoris strains that produce glycoproteins having predominantly NANA₂Gal₂GlcNAc₂Man₃GlcNAc₂ N-glycans.

The term “GS5.0”, when used herein refers to the N-glycosylation structure Gal₂GlcNAc₂Man₃ GlcNAc₂.

The term “GS5.5”, when used herein refers to the N-glycosylation structure NANAGal₂GlcNAc₂Man₃GlcNAc₂, which when produced in Pichia pastoris strains to which α 2,6 sialyl transferase has been glycoengineered result in α-2,6-linked sialic acid and which when produced in Pichia pastoris strains to which α-2,3 sialyl transferase has been glycoengineered result in α-2,3-linked sialic acid.

The term “GS6.0”, when used herein refers to the N-glycosylation structure NANA₂Gal₂GlcNAc₂Man₃GlcNAc₂, which when produced in Pichia pastoris strains to which α-2,6 sialyltransferase has been glycoengineered result in α-2,6-linked sialic acid and which when produced in Pichia pastoris strains to which α-2,3 sialyl transferase has been glycoengineered result in α-2,3-linked sialic acid.

The term “wild type” or “wt” when used herein in connection to a Pichia pastoris strain refers to a native Pichia pastoris strain that has not been subjected to genetic modification to control glycosylation.

The term “wild type” or “wt” when used herein in connection to an FcR refers to a an FcR polypeptode or amino acid that comprises the a native nucleotide or amino acid sequence—a sequence that has not been subject to any genetic modification.

The terms “N-glycan”, “glycoprotein” and “glycoform” when used herein refer to an N-linked oligosaccharide, e.g., one that is attached by an asparagine-N-acetylglucosamine linkage to an asparagine residue of a polypeptide. Predominant sugars found on glycoproteins are glucose, galactose, mannose, fucose, N-acetylgalactosamine (GalNAc), N-acetylglucosamine (GlcNAc) and sialic acid (SA, including NANA, NGNA and derivatives and analogs thereof, including acetylated NANA or acetylated NGNA). In glycoengineered Pichia pastoris, sialic acid is exclusively N-acetyl-neuraminic acid (NANA) (Hamilton et al., Science 313 (5792): 1441-1443 (2006)). N-glycans have a common pentasaccharide core of Man₃GlcNAc₂, wherein “Man” refers to mannose, “Glc” refers to glucose, “NAc” refers to N-acetyl, and GlcNAc refers to N-acetylglucosamine. N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid).

As used herein, the term “sialic acid” or “SA” refers to any member of the sialic acid family, including without limitation: N-acetylneuraminic acid (NeuSAc or NANA), N-glycolylneuraminic acid (NGNA) and any analog or derivative thereof (including those arising from acetylation at any position on the sialic acid molecule). Sialic acid is a generic name for a group of about 30 naturally occurring acidic carbohydrates that are essential components of a large number of glycoconjugates. Schauer, Biochem. Society Transactions, 11, 270-271 (1983). Sialic acids are usually the terminal residue of the oligosaccharides. N-acetylneuraminic acid (NANA) is the most common sialic acid form and N-glycolylneuraminic acid (NGNA) is the second most common form. Schauer, Glycobiology, 1, 449-452 (1991). NGNA is widespread throughout the animal kingdom and, according to species and tissue, often constitutes a significant proportion of the glycoconjugate-bound sialic acid. Certain species such as chicken and man are exceptional, since they lack NGNA in normal tissues. Corfield, et al., Cell Biology Monographs, 10, 5-50 (1982). In human serum samples, the percentage of sialic acid in the form of NGNA is reported to be 0.01% of the total sialic acid. Schauer, “Sialic Acids as Antigenic Determinants of Complex Carbohydrates”, found in The Molecular Immunology of Complex Carbohydrates, (Plenum Press, New York, 1988).

The term “human-like N-glycan”, as used herein, refers to the N-linked oligosaccharides which closely resemble the oligosaccharides produced by non-engineered, wild-type human cells. For example, wild-type Pichia pastoris and other lower eukaryotic cells typically produce hypermannosylated proteins at N-glycosylation sites. The host cells described herein produce glycoproteins (for example, antibodies) comprising human-like N-glycans that are not hypermannosylated. In some embodiments, the host cells of the present invention are capable of producing human-like N-glycans with hybrid and/or complex N-glycans. The specific type of “human-like” glycans present on a specific glycoprotein produced from a host cell of the invention will depend upon the specific glycoengineering steps that are performed in the host cell.

The term “high mannose” type N-glycan when used herein refers to an N-glycan having five or more mannose residues.

The term “complex” type N-glycan when used herein refers to an N-glycan having at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). As an example, when a N-glycan comprises a bisecting GlcNAc on the trimannose core, the structure can be represented as Man₃GlcNAc₂(GlcNAc) or Man₃GlcNAc₃. When an N-glycan comprises a core fucose attached to the trimannose core, the structure may be represented as Man₃GlcNAc₂(Fuc). Complex N-glycans may also have multiple antennae on the “trimannose core,” often referred to as “multiple antennary glycans.”

The term “hybrid” N-glycan when used herein refers to an N-glycan having at least one GlcNAc on the terminal of the 1,3 mannose arm of the trimannose core and zero or more than one mannose on the 1,6 mannose arm of the trimannose core.

When referring to “mole percent” of a glycan present in a preparation of a glycoprotein, the term means the molar percent of a particular glycan present in the pool of N-linked oligosaccharides released when the protein preparation is treated with PNGase and then quantified by a method that is not affected by glycoform composition, (for instance, labeling a PNGase released glycan pool with a fluorescent tag such as 2-aminobenzamide and then separating by high performance liquid chromatography or capillary electrophoresis and then quantifying glycans by fluorescence intensity). For example, 50 mole percent NANA₂Gal₂GlcNAc₂Man₃GlcNAc₂ means that 50 percent of the released glycans are NANA₂Gal₂GlcNAc₂Man₃GlcNAc₂ and the remaining 50 percent are comprised of other N-linked oligosaccharides. Thus, in this application, the terms “mole percent” and “percent” are used interchangeably.

The term “anti-inflammatory antibody” as used herein, refers to an antibody intended to be used to treat inflammation. The anti-inflammatory properties of an Fc-containing polypeptide can be measured using any method known in the art. In one embodiment, the anti-inflammatory properties of an Fc-containing polypeptide are measured using an animal model, such as the models described in Kaneko et al., Science 313:670-673 (2006), Anthony et al., Science 320:373-376 (2008), WO2011/149999 and WO2013/095966. In another embodiment, the anti-inflammatory properties of an Fc-containing polypeptide are measured by determining the level of a biomarker related to inflammation (including without limitation: CRP, pro-inflammatory cytokines such as tumor necrosis factors (TNF-alpha), interferon-gamma, interleukin 6 (IL-6, IL-8, IL-10, chemokines, the coagulation marker D-dimer, sCD14, intestinal fatty acid binding peptide (IFABP), and hyaluronic acid. In one embodiment, the anti-inflammatory properties of an Fc-containing polypetpide is measured by determining the level of C-reactive protein (CRP) using a method known in the art. A decrease in the level of C-reactive protein indicates that the Fc-containing polypeptide has anti-inflammatory properties.

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity of the protein. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are listed below:

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

Glycosylation of immunoglobulin G (IgG) in the Fc region, Asn297 (according to the EU numbering system), has been shown to be a requirement for optimal recognition and activation of effector pathways including antibody dependent cellular cytotoxicity (ADCC) and complement dependent cytotoxicity (CDC), Wright & Morrison, Trends in Biotechnology, 15: 26-31 (1997), Tao & Morrison, J. Immunol., 143(8):2595-2601 (1989). As such, glycosylation engineering in the constant region of IgG has become an area of active research for the development of therapeutic monoclonal antibodies (mAbs). It has been established that the presence of N-linked glycosylation at Asn297 is critical for mAb activity in immune effector function assays including ADCC, Rothman (1989), Lifely et al., Glycobiology, 5:813-822 (1995), Umana (1999), Shields (2002), and Shinkawa (2003), and complement dependent cytotoxicity (CDC), Hodoniczky et al., Biotechnol. Prog., 21(6): 1644-1652 (2005), and Jefferis et al., Chem. Immunol., 65: 111-128 (1997). This effect on function has been attributed to the specific conformation adopted by the glycosylated Fc domain, which appears to be lacking when glycosylation is absent. More specifically, IgG which lacks glycosylation in the Fc C_{H}2 domain does not bind to FcγR, including FcγRI, FcγRII, and FcγRIII, Rothman (1989).

Not only does the presence of glycosylation appear to play a role in the effector function of an antibody, the particular composition of the N-linked oligosaccharide is also important. For example, the presence of fucose shows a marked effect on in vitro FcγRIIIa binding and in vitro ADCC, Rothman (1989), and Li et al., Nat. Biotechnol. 24(2): 2100-215 (2006). Recombinant antibodies produced by mammalian cell culture, such as CHO or NS0, contain N-linked oligosaccharides that are predominately fucosylated, Hossler et al., Biotechnology and Bioengineering, 95(5):946-960 (2006), Umana (1999), and Jefferis et al., Biotechnol. Prog. 21:11-16 (2005). Additionally, there is evidence that sialylation in the Fc region may impart anti-inflammatory properties to antibodies. Intravenous immunoglobulin (WIG) purified over a lectin column to enrich for the sialylated form showed a distinct anti-inflammatory effect limited to the sialylated Fc fragment and was linked to an increase in expression of the inhibitory receptor FcγRIIb, Nimmerjahn and Ravetch., J. Exp. Med. 204:11-15 (2007).

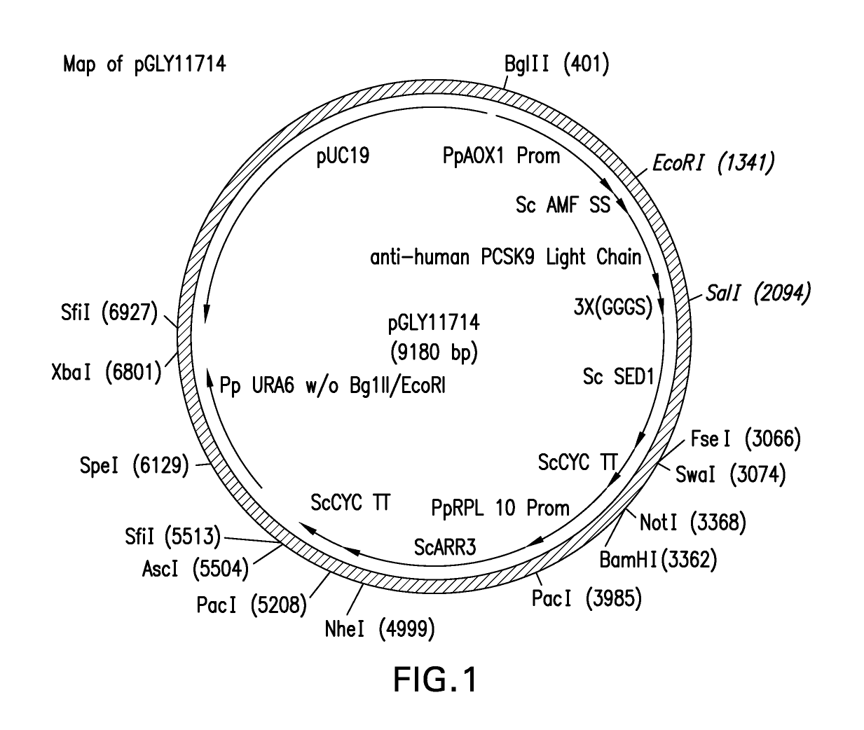

Glycosylation in the Fc region of an antibody derived from mammalian cell lines typically consists of a heterogeneous mix of glycoforms, with the predominant forms typically being comprised of the complex fucosylated glycoforms: G0F, G1F, and, to a lesser extent, G2F. Possible conditions resulting in incomplete galactose transfer to the G0F structure include, but are not limited to, non-optimized galactose transfer machinery, such as β-1,4 galactosyl transferase, and poor UDP-galactose transport into the Golgi apparatus, suboptimal cell culture and protein expression conditions, and steric hindrance by amino acid residues neighboring the oligosaccharide. While each of these conditions may modulate the ultimate degree of terminal galactose, it is thought that subsequent sialic acid transfer to the Fc oligosaccharide is inhibited by the closed pocket configuration of the C_{H}2 domain. See, for example, FIG. 1, Jefferis, R., Nature Biotech., 24 (10): 1230-1231, 2006. Without the correct terminal monosaccharide, specifically galactose, or with insufficient terminal galactosylated forms, there is little possibility of producing a sialylated form, capable of acting as a therapeutic protein, even when produced in the presence of sialyl transferase. Protein engineering and structural analysis of human IgG-Fc glycoforms has shown that glycosylation profiles are affected by Fc conformation, such as the finding that increased levels of galactose and sialic acid on oligosaccharides derived from CHO-produced IgG3 could be achieved when specific amino acids from the Fc pocket were mutated, to an alanine including F241, F243, V264, D265, Y296 and R301. Lund et al., J. Immunol. 157(11); 4963-4969 (1996). It was further shown that certain mutations had some effect on cell mediated superoxide generation and complement mediated red cell lysis, which are used as surrogate markers for FcγRI and Clq binding, respectively.

Yeast have been genetically engineered to produce host strains capable of secreting glycoproteins with highly uniform glycosylation. Choi et al., PNAS, USA 100(9): 5022-5027 (2003) describes the use of libraries of α 1,2 mannosidase catalytic domains and N-acetylglucosaminyltransferase I catalytic domains in combination with a library of fungal type II membrane protein leader sequences to localize the catalytic domains to the secretory pathway. In this way, strains were isolated that produced in vivo glycoproteins with uniform Man₅GlcNAc₂ or GlcNAcMan₅GlcNAc₂ N-glycan structures. Hamilton et al., Science 313 (5792): 1441-1443 (2006) described the production of a glycoprotein, erythropoietin, produced in Pichia pastoris, as having a glycan composition that consisted predominantly of a bisialylated glycan structure, GS6.0, NANA₂Gal₂GlcNAc₂Man₃GlcNAc₂ (90.5%) and monosialylated, GS5.5, NANAGal₂GlcNAc₂ Man₃GlcNAc₂ (7.9%). WO201//149999 describes the production of sialyulated antibodies in Pichia pastoris.

### Host Organisms and Cell Lines

The Fc-containing polypeptides of this invention can be made in any host organism or cell line.

In one embodiment, an Fc-containing polypeptide of the invention is made in a host cell which is capable of producing proteins comprising N-glycans.

In one embodiment, an Fc-containing polypeptide of the invention is made in a host cell which is capable of producing proteins comprising sialylated N-glycans.

In one embodiment, an Fc-containing polypeptide of the invention is made in a mammalian cell where the cell either endogenously or through genetic or process manipulation produces glycoproteins containing either a mixture of terminal α2-6 and α2-3 sialic acid, or only terminal α2-6 sialic acid. The propagation of mammalian cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO); mouse sertoli cells (TM4,); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; hybridoma cell lines; NS0; SP2/0; and a human hepatoma line (Hep G2).

In one embodiment, an Fc-containing polypeptide of the invention can be made in a plant cell which is engineered to produce sialylated N-glycans. See, e.g., Cox et al., Nature Biotechnology (2006) 24, 1591-1597 (2006) and Castilho et al., J. Biol. Chem. 285(21): 15923-15930 (2010).

In one embodiment, an Fc-containing polypeptide of the invention can be made in an insect cell which is engineered to produce sialylated N-glycans. See, e.g., Harrison and Jarvis, Adv. Virus Res. 68:159-91 (2006).

In one embodiment, an Fc-containing polypeptide of the invention can be made in a bacterial cell which is engineered to produce sialylated N-glycans. See, e.g., Lizak et al., Bioconjugate Chem. 22:488-496 (2011).

### Lower Eukaryotic Organisms as Host Cells

In one embodiment, an Fc-containing polypeptide of the invention can be made in a lower eukaryotic host cell or organism. Recent developments allow the production of fully humanized therapeutics in lower eukaryotic host organisms, yeast and filamentous fungi, such as Pichia pastoris, Gerngross et al., U.S. Pat. No. 7,029,872 and U.S. Pat. No. 7,449,308, the disclosures of which are hereby incorporated by reference. See also Jacobs et al., Nature Protocols 4(1):58-70 (2009).

In one embodiment, an Fc-containing polypeptide of the invention is made in a lower eukaryotic host cell, more preferably a yeast or filamentous fungal host cell, that has been engineered to produce glycoproteins comprising human-like N-glycans. In another embodiment, an Fc-containing polypeptide of the invention is made in a lower eukaryotic host cell, more preferably a yeast or filamentous fungal host cell, that has been engineered to produce glycoproteins having a predominant N-glycan comprising a terminal sialic acid. In one embodiment of the invention, the predominant N-glycan is the α-2,6 linked form of SA₂Gal₂GlcNAc₂Man₃GlcNAc₂, produced in strains glycoengineered with α-2,6 sialyl transferase which do not produce any α-2,3 linked sialic acid.

Those of ordinary skill in the art would recognize and appreciate that the materials and methods described herein are not limited to the specific strain of Pichia pastoris provided as an example herein, but could include any Pichia pastoris strain or other yeast or filamentous fungal strains capable of producing human-like N-glycans.

In general, lower eukaryotes such as yeast are used for expression of the proteins, particularly glycoproteins because they can be economically cultured, give high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

While the invention has been demonstrated herein using the methylotrophic yeast Pichia pastoris, other useful lower eukaryote host cells include Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporiumi lucknowense, Fusarium sp., Fusarium gramineum, Fusarium venenatum and Neurospora crassa. Various yeasts, such as K. lactis, Pichia pastoris, Pichia methanolica, and Hansenula polymorpha are particularly suitable for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Lower eukaryotes, particularly yeast and filamentous fungi, can be genetically modified so that they express glycoproteins in which the glycosylation pattern is human-like or humanized. As indicated above, the term “human-like N-glycan”, as used herein refers, to the N-linked oligosaccharides which closely resemble the oligosaccharides produced by non-engineered, wild-type human cells. In preferred embodiments of the present invention, the host cells of the present invention are capable of producing human-like glycoproteins with hybrid and/or complex N-glycans; i.e., “human-like N-glycosylation.” The specific “human-like” glycans predominantly present on glycoproteins produced from the host cells of the invention will depend upon the specific engineering steps that are performed. In this manner, glycoprotein compositions can be produced in which a specific desired glycoform is predominant in the composition. Such can be achieved by eliminating selected endogenous glycosylation enzymes and/or genetically engineering the host cells and/or supplying exogenous enzymes to mimic all or part of the mammalian glycosylation pathway as described in U.S. Pat. No. 7,449,308. If desired, additional genetic engineering of the glycosylation can be performed, such that the glycoprotein can be produced with or without core fucosylation. Use of lower eukaryotic host cells is further advantageous in that these cells are able to produce highly homogenous compositions of glycoprotein, such that the predominant glycoform of the glycoprotein may be present as greater than thirty mole percent of the glycoprotein in the composition. In particular aspects, the predominant glycoform may be present in greater than forty mole percent, fifty mole percent, sixty mole percent, seventy mole percent and, most preferably, greater than eighty mole percent of the glycoprotein present in the composition.

Lower eukaryotes, particularly yeast, can be genetically modified so that they express glycoproteins in which the glycosylation pattern is human-like or humanized. Such can be achieved by eliminating selected endogenous glycosylation enzymes and/or supplying exogenous enzymes as described by Gerngross et al., U.S. Pat. No. 7,449,308. For example, a host cell can be selected or engineered to be depleted in α1,6-mannosyl transferase activities, which would otherwise add mannose residues onto the N-glycan on a glycoprotein.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. Nos. 7,029,872 and 7,449,308 and U.S. Published Patent Application No. 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAcMan₅GlcNAc₂ glycoform. The glycoprotein produced in the above cells can be treated in vitro with a hexosaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAcMan₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. Nos. 7,029,872 and 7,449,308 and U.S. Published Patent Application No. 2005/0170452 disclose lower eukaryote host cells capable of producing a glycoprotein comprising a GlcNAc₂Man₃GlcNAc₂ glycoform. The glycoprotein produced in the above cells can be treated in vitro with a hexosaminidase to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂ Man₃GlcNAc₂ or Gal₂GlcNAc₂Man₃GlcNAc₂ glycoform, or mixture thereof for example a recombinant glycoprotein composition comprising predominantly a GalGlcNAc₂ Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353 discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃ GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialyltransferase activity to the ER or Golgi apparatus of the host cell. In a preferred embodiment, the sialyltransferase is an α-2,6-sialyltransferase. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. To enhance the amount of sialylation, it can be advantageous to construct the host cell to include two or more copies of the CMP-sialic acid synthesis pathway or two or more copies of the sialylatransferase. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2005/0208617 and 2007/0037248. Further, the proceeding host cells can produce recombinant glycoproteins (for example, antibodies) comprising SA(1-4)Gal(1-4)GlcNAc(2-4) Man₃GlcNAc₂, including antibodies comprising NANA (1-4)Gal(1-4)GlcNAc(2-4) Man₃GlcNAc₂, NGNA(1-4)Gal(1-4)GlcNAc(2-4)Man₃GlcNAc₂ or a combination of NANA (1-4)Gal(1-4)GlcNAc(2-4) Man₃GlcNAc₂ and NGNA(1-4)Gal(1-4)GlcNAc(2-4) Man₃GlcNAc₂. In one embodiment, the recombinant glycoprotein will comprise N-glycans comprising a structure selected from the group consisting of SA(1-4)Gal(1-4)GlcNAc(2-4) Man₃GlcNAc₂ and devoid of any α2-3 linked SA.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target the galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the GalGlcNAcMan₅GlcNAc₂ N-glycans further includes a sialyltransferase catalytic domain fused to a cellular targeting signal peptide not normally associated with the catalytic domain and selected to target sialyltransferase activity to the ER or Golgi apparatus of the host cell. Passage of the recombinant glycoprotein through the ER or Golgi apparatus of the host cell produces a recombinant glycoprotein comprising a SAGalGlcNAcMan₅GlcNAc₂ glycoform (for example NANAGalGlcNAcMan₅GlcNAc₂ or NGNAGalGlcNAcMan₅GlcNAc₂ or a mixture thereof).

Any of the preceding host cells can further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

Further, any of the preceding host cells can be further manipulated to increase N-glycan occupancy. See e, g., Gaulitzek et al., Biotechnol. Bioengin. 103:1164-1175 (2009); Jones et al., Biochim. Biospyhs. Acta 1726:121-137 (2005); WO2006/107990. In one embodiment, any of the preceding host cells can be further engineered to comprise at least one nucleic acid molecule encoding a heterologous single-subunit oligosaccharyltransferase (for example, Leishmania sp. STT3A protein, STT3B protein, STT3C protein, STT3D protein or combinations thereof) and a nucleic acid molecule encoding the heterologous glycoprotein, and wherein the host cell expresses the endogenous host cell genes encoding the proteins comprising the endogenous OTase complex. In one embodiment, any of the preceding host cells can be further engineered to comprise at least one nucleic acid molecule encoding a Leishmania sp. STT3D protein and a nucleic acid molecule encoding the heterologous glycoprotein, and wherein the host cell expresses the endogenous host cell genes encoding the proteins comprising the endogenous OTase complex.

Host cells further include lower eukaryote cells (e.g., yeast such as Pichia pastoris) that are genetically engineered to produce glycoproteins that do not have α-mannosidase-resistant N-glycans. This can be achieved by deleting or disrupting one or more of the β-mannosyltransferase genes (e.g., BMT1, BMT2, BMT3, and BMT4) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007), which in further aspects can also include deleting or disrupting the MNN4A gene. Disruption includes disrupting the open reading frame encoding the particular enzymes or disrupting expression of the open reading frame or abrogating translation of RNAs encoding one or more of the β-mannosyltransferases and/or phosphomannosyltransferases using interfering RNA, antisense RNA, or the like. Further, cells can produce glycoproteins with α-mannosidase-resistant N-glycans through the addition of chemical hinhibios or through modification of the cell culture condition. These host cells can be further modified as described above to produce particular N-glycan structures.

Host cells further include lower eukaryote cells (e.g., yeast such as Pichia pastoris) that are genetically modified to control O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377) or grown in the presence of Pmtp inhibitors and/or an α-mannosidase as disclosed in Published International Application No. WO 2007/061631, or both. Disruption includes disrupting the open reading frame encoding the Pmtp or disrupting expression of the open reading frame or abrogating translation of RNAs encoding one or more of the Pmtps using interfering RNA, antisense RNA, or the like. The host cells can further include any one of the aforementioned host cells modified to produce particular N-glycan structures.

Pmtp inhibitors include but are not limited to a benzylidene thiazolidinediones. Examples of benzylidene thiazolidinediones that can be used are 5-[[3,4-bis(phenylmethoxy) phenyl]methylene]-4-oxo-2-thioxo-3-thiazolidineacetic Acid; 5-[[3-(1-Phenylethoxy)-4-(2-phenylethoxy)]phenyl]methylene]-4-oxo-2-thioxo-3-thiazolidineacetic Acid; and 5-[[3-(1-Phenyl-2-hydroxy)ethoxy)-4-(2-phenylethoxy)]phenyl]methylene]-4-oxo-2-thioxo-3-thiazolidineacetic acid.

In particular embodiments, the function or expression of at least one endogenous PMT gene is reduced, disrupted, or deleted. For example, in particular embodiments the function or expression of at least one endogenous PMT gene selected from the group consisting of the PMT1, PMT2, PMT3, and PMT4 genes is reduced, disrupted, or deleted; or the host cells are cultivated in the presence of one or more PMT inhibitors. In further embodiments, the host cells include one or more PMT gene deletions or disruptions and the host cells are cultivated in the presence of one or more Pmtp inhibitors. In particular aspects of these embodiments, the host cells also express a secreted α-1,2-mannosidase.

PMT deletions or disruptions and/or Pmtp inhibitors control O-glycosylation by reducing O-glycosylation occupancy, that is, by reducing the total number of O-glycosylation sites on the glycoprotein that are glycosylated. The further addition of an α-1,2-mannsodase that is secreted by the cell controls O-glycosylation by reducing the mannose chain length of the O-glycans that are on the glycoprotein. Thus, combining PMT deletions or disruptions and/or Pmtp inhibitors with expression of a secreted α-1,2-mannosidase controls O-glycosylation by reducing occupancy and chain length. In particular circumstances, the particular combination of PMT deletions or disruptions, Pmtp inhibitors, and α-1,2-mannosidase is determined empirically as particular heterologous glycoproteins (Fabs and antibodies, for example) may be expressed and transported through the Golgi apparatus with different degrees of efficiency and thus may require a particular combination of PMT deletions or disruptions, Pmtp inhibitors, and α-1,2-mannosidase. In another aspect, genes encoding one or more endogenous mannosyltransferase enzymes are deleted. This deletion(s) can be in combination with providing the secreted α-1,2-mannosidase and/or PMT inhibitors or can be in lieu of providing the secreted α-1,2-mannosidase and/or PMT inhibitors.

Thus, the control of O-glycosylation can be useful for producing particular glycoproteins in the host cells disclosed herein in better total yield or in yield of properly assembled glycoprotein. The reduction or elimination of O-glycosylation appears to have a beneficial effect on the assembly and transport of whole antibodies and Fab fragments as they traverse the secretory pathway and are transported to the cell surface. Thus, in cells in which O-glycosylation is controlled, the yield of properly assembled antibodies or Fab fragments is increased over the yield obtained in host cells in which O-glycosylation is not controlled.

To reduce or eliminate the likelihood of N-glycans and O-glycans with β-linked mannose residues, which are resistant to α-mannosidases, the recombinant glycoengineered Pichia pastoris host cells are genetically engineered to eliminate glycoproteins having α-mannosidase-resistant N-glycans by deleting or disrupting one or more of the β-mannosyltransferase genes (e.g., BMT1, BMT2, BMT3, and BMT4) (See, U.S. Pat. No. 7,465,577 and U.S. Pat. No. 7,713,719). The deletion or disruption of BMT2 and one or more of BMT1, BMT3, and BMT4 also reduces or eliminates detectable cross reactivity to antibodies against host cell protein.

Yield of glycoprotein can in some situations be improved by overexpressing nucleic acid molecules encoding mammalian or human chaperone proteins or replacing the genes encoding one or more endogenous chaperone proteins with nucleic acid molecules encoding one or more mammalian or human chaperone proteins. In addition, the expression of mammalian or human chaperone proteins in the host cell also appears to control O-glycosylation in the cell. Thus, further included are the host cells herein wherein the function of at least one endogenous gene encoding a chaperone protein has been reduced or eliminated, and a vector encoding at least one mammalian or human homolog of the chaperone protein is expressed in the host cell. Also included are host cells in which the endogenous host cell chaperones and the mammalian or human chaperone proteins are expressed. In further aspects, the lower eukaryotic host cell is a yeast or filamentous fungi host cell. Examples of the use of chaperones of host cells in which human chaperone proteins are introduced to improve the yield and reduce or control O-glycosylation of recombinant proteins has been disclosed in Published International Application No. WO 2009105357 and WO2010019487 (the disclosures of which are incorporated herein by reference). Like above, further included are lower eukaryotic host cells wherein, in addition to replacing the genes encoding one or more of the endogenous chaperone proteins with nucleic acid molecules encoding one or more mammalian or human chaperone proteins or overexpressing one or more mammalian or human chaperone proteins as described above, the function or expression of at least one endogenous gene encoding a protein O-mannosyltransferase (PMT) protein is reduced, disrupted, or deleted. In particular embodiments, the function of at least one endogenous PMT gene selected from the group consisting of the PMT1, PMT2, PMT3, and PMT4 genes is reduced, disrupted, or deleted.

In addition, O-glycosylation may have an effect on an antibody or Fab fragment's affinity and/or avidity for an antigen. This can be particularly significant when the ultimate host cell for production of the antibody or Fab is not the same as the host cell that was used for selecting the antibody. For example, O-glycosylation might interfere with an antibody's or Fab fragment's affinity for an antigen, thus an antibody or Fab fragment that might otherwise have high affinity for an antigen might not be identified because O-glycosylation may interfere with the ability of the antibody or Fab fragment to bind the antigen. In other cases, an antibody or Fab fragment that has high avidity for an antigen might not be identified because O-glycosylation interferes with the antibody's or Fab fragment's avidity for the antigen. In the preceding two cases, an antibody or Fab fragment that might be particularly effective when produced in a mammalian cell line might not be identified because the host cells for identifying and selecting the antibody or Fab fragment was of another cell type, for example, a yeast or fungal cell (e.g., a Pichia pastoris host cell). It is well known that O-glycosylation in yeast can be significantly different from O-glycosylation in mammalian cells. This is particularly relevant when comparing wild type yeast O-glycosylation with mucin-type or dystroglycan type O-glycosylation in mammals. In particular cases, O-glycosylation might enhance the antibody or Fab fragments affinity or avidity for an antigen instead of interfere with antigen binding. This effect is undesirable when the production host cell is to be different from the host cell used to identify and select the antibody or Fab fragment (for example, identification and selection is done in yeast and the production host is a mammalian cell) because in the production host the O-glycosylation will no longer be of the type that caused the enhanced affinity or avidity for the antigen. Therefore, controlling O-glycosylation can enable use of the materials and methods herein to identify and select antibodies or Fab fragments with specificity for a particular antigen based upon affinity or avidity of the antibody or Fab fragment for the antigen without identification and selection of the antibody or Fab fragment being influenced by the O-glycosylation system of the host cell. Thus, controlling O-glycosylation further enhances the usefulness of yeast or fungal host cells to identify and select antibodies or Fab fragments that will ultimately be produced in a mammalian cell line.

Those of ordinary skill in the art would further appreciate and understand how to utilize the methods and materials described herein in combination with other Pichia pastoris and yeast cell lines that have been genetically engineered to produce specific N-glycans or sialylated glycoproteins, such as, but, not limited to, the host organisms and cell lines described above that have been genetically engineered to produce specific galactosylated or sialylated forms. See, for example, US 2006-0286637, Production of Sialylated N-Glycans in Lower Eukaryotes, in which the pathway for galactose uptake and utilization as a carbon source has been genetically modified, the description of which is incorporated herein as if set forth at length. See also WO2011/149999.

### Biological Properties of Fc Muteins

For many Fc-containing polypeptides increased anti-inflammatory properties would be desirable characteristics. In any of the embodiments of the invention, an increase in anti-inflammatory activity can be detected using any method known in the art. In one embodiment, an increase in anti-inflammatory activity is detected by measuring a decrease in the expression of a gene selected from the group consisting of: IL-1β, IL-6, RANKL, TRAP, ATP6v0d2, MDL-1, DAP12, CD11b, TIMP-1, MMP9, CTSK, PU-1, MCP1, MIP1α, Cxcl1-Groa, CXcl2-Grob, CD18, TNF, FcγRI, FcγRIIb, FcγRIII and FcγRIV.

### Production of Fc-Containing Polypeptides

The Fc-containing polypeptides of the invention can be made according to any method known in the art suitable for generating polypeptides comprising N-glycans. In one embodiment, the Fc-containing polypeptide is an antibody or an antibody fragment (including, without limitation a polypeptide consisting of or consisting essentially of the Fc region of an antibody). In one embodiment, the antibody or antibody fragment is of the IgG type. In one embodiment, the antibody or antibody fragment is of the IgG1 subtype. In one embodiment, the antibody or antibody fragment is of the IgG2 subtype. In one embodiment, the antibody or antibody fragment is of the IgG3 subtype. In one embodiment, the antibody or antibody fragment is of the IgG4 subtype. In one embodiment, the antibody or antibody fragment is of the IgA type. In one embodiment, the antibody or antibody fragment is of the IgD type. In one embodiment, the antibody or antibody fragment is of the IgE type. In one embodiment, the antibody or antibody fragment is of the IgM type. In another embodiment, the Fc-containing polypeptide is an immunoadhesin. Methods of preparing antibody and antibody fragments are well known in the art. Methods of introducing point mutations into a polypeptide, for example site directed mutagenesis, are also well known in the art.

In one embodiment, the Fc-containing polypeptide comprises N-glycans. In one embodiment, the Fc-containing polypeptide of the invention comprises N-glycans, wherein at least 30%, 40%, 50%, 60%, 70%, 80% or 90% of the N-glycans on the Fc-containing polypeptide comprise an N-linked oligosaccharide structure selected from the group consisting of SA₍₁₋₄₎Gal₍₁₋₄₎GlcNAc₍₂₋₄₎Man₃GlcNAc₂. In one embodiment, at least 30%, 40%, 50%, 60%, 70%, 80% or 90% of the N-glycans on the Fc-containing polypeptide comprise a SA₂Gal₂GlcNAc₂Man₃GlcNAc structure. In one embodiment, at least 30%, 40%, 50%, 60%, 70%, 80% or 90% of the N-glycans on the Fc-containing polypeptide comprise a NANA₂Gal₂GlcNAc₂Man₃GlcNAc structure.

### N-Glycan Analysis of Fc Muteins

For many glycoproteins, including certain antibodies, N-glycosylation is important for biological properties. For example, sialylation of the terminal N-linked glycan of an IgG Fc region is important for producing glycoproteins and antibodies that have the correct conformation to impart therapeutic activity. See, for example, Anthony et al., Science, 320: 373-376 (2008), where terminal sialylation was correlated to anti-inflammatory activity for an WIG preparation. Sialylation requires the presence of a penultimate galactose, upon which the sialyl transferase acts to form the sialylated glycan.

The N-glycan composition of the antibodies can be analyzed by matrix-assisted laser desorption ionization/time-of-flight (MALDI-TOF) mass spectrometry after release from the antibody with peptide-N-glycosidase F. Released carbohydrate composition can be quantitated by HPLC on an Allentech Prevail carbo (Alltech Associates, Deerfield Ill.) column.

### FcγR and FcRn Binding of Fc Muteins

The FcγR and FcRn binding of Fc muteins can be determined using any method known in the art. See, e.g., Shields et al., J. Biol. Chem. 276: 6591-6604 (2001); Vaccaro et al., Nat Biotechnol. 23(10):1283-8 (2005).

### Biological Targets

Those of ordinary skill in the art would recognize and appreciate that the materials and methods herein could be used to produce any Fc-containing polypeptide for which the characteristics of enhanced anti-inflammatory activity or decreased effector function would be desirable. It should further be noted that there is no restriction as to the type of Fc-containing polypeptide or antibody so produced by the invention. The Fc region of the Fc-containing polypeptide could be from an IgA, IgD, IgE, IgG or IgM. In one embodiment, the Fc region of the Fc-containing polypeptide is from an IgG, including IgG1, IgG2, IgG3 or IgG4. In one embodiment, Fc region of the Fc-containing polypeptide is from an IgG1. In specific embodiments the antibodies or antibody fragments produced by the materials and methods herein can be chimeric, humanized or human antibodies.

In some embodiments, the Fc-containing polypeptides of the invention will bind to a biological target that is involved in inflammation.

In some embodiments, the Fc-containing polypeptide of the invention will bind to a pro-inflammatory cytokine. In some embodiments, the Fc-containing polypeptide of the invention will bind to a molecule selected from the group consisting of: TNF-α, IL-1, IL-2, IL-4, IL-5, IL-6, IL-8, IL-9, IL-10, IL-12, IL-15, IL-17, IL-18, IL-20, IL-21, IL-22, IL-23, IL-23R, IL-25, IL-27, IL-33, CD2, CD4, CD11A, CD14, CD18, CD19, CD23, CD25, CD40, CD40L, CD20, CD52, CD64, CD80, CD147, CD200, CD200R, TSLP, TSLPR, PD-1, PDL1, CTLA4, VLA-4, VEGF, PCSK9, α4β7-integrin, E-selectin, Fact II, ICAM-3, beta2-integrin, IFNγ, C5, CBL, LCAT, CR3, MDL-1, GITR, ADDL, CGRP, TRKA, IGF1R, RANKL, GTC, αBLys, or the receptor for any of the above mentioned molecules. In one embodiment, the Fc-containing polypeptide of the invention will bind to TNF-α. In another embodiment, the Fc-containing polypeptide of the invention will bind to Her2. In another embodiment, the Fc-containing polypeptide of the invention will bind to PCSK9. In another embodiment, the Fc-containing polypeptide of the invention will bind to TNFR. In another embodiment, the Fc-containing polypeptide of the invention will bind to LCAT. In another embodiment, the Fc-containing polypeptide of the invention will bind to TSLP. In another embodiment, the Fc-containing polypeptide of the invention will bind to PD-1. In another embodiment, the Fc-containing polypeptide of the invention will bind to IL-23. In another embodiment, the Fc-containing polypeptide of the invention will bind to αBLys.

In some embodiments, the Fc-containing polypeptides of the invention will be specific for an antigen selected from autoimmune antigens, allergens, MHC molecules or Rhesus factor D antigen. See, e.g., the antigens listed in Table 1 of WO2010/10910, which is incorporated herein by reference.

### Methods of Increasing Anti-Inflammatory Properties

The invention also comprises a method of increasing the anti-inflammatory properties of an Fc-containing polypeptide comprising: selecting a parent Fc-containing polypeptide that is useful in treating an inflammatory condition (for example, an antibody or immunoadhesin that binds to an antigen that is involved in inflammation) and introducing one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region in the Fc-containing polypeptide, wherein the numbering is according to the EU index as in Kabat, wherein the Fc-containing polypeptide has increased anti-inflammatory properties when compared to the parent Fc-containing polypeptide. In one embodiment, the Fc-containing polypeptide comprises N-glycans. In one embodiment, the Fc-containing polypepeptide comprises sialylated N-glycans. In one embodiment, the parent Fc-containing polypeptide is an antibody, antibody fragment or immunoadhesin that binds to an antigen that is involved in inflammation.

In one embodiment, the parent Fc-containing polypeptide is an an antibody, antibody fragment or immunoadhesin that is already marketed or under development for the treatment of an inflammatory conditions. In another embodiment, the parent Fc-containing polypeptide is an antibody selected from the group consisting of: Muromonab-CD3 (anti-CD3 receptor antibody), Abciximab (anti-CD41 7E3 antibody), Rituximab (anti-CD20 antibody), Daclizumab (anti-CD25 antibody), Basiliximab (anti-CD25 antibody), Palivizumab (anti-RSV (respiratory syncytial virus) antibody), Infliximab (anti-TNFα antibody), Trastuzumab (anti-Her2 antibody), Gemtuzumab ozogamicin (anti-CD33 antibody), Alemtuzumab (anti-CD52 antibody), Ibritumomab tiuxeten (anti-CD20 antibody), Adalimumab (anti-TNFα antibody), Omalizumab (anti-IgE antibody), Tositumomab-131I (iodinated derivative of an anti-CD20 antibody), Efalizumab (anti-CD11a antibody), Cetuximab (anti-EGF receptor antibody), Golimumab (anti-TNFα antibody), Bevacizumab (anti VEGF-A antibody), Natalizumab (anti α4 integrin), Efalizumab (anti CD11a), Cetolizumab (anti-TNFα antibody), Tocilizumab (anti-IL-6R), Ustenkinumab (anti IL-12/23), alemtuzumab (anti CD52), and natalizumab (anti α4 integrin), and variants thereof. In another embodiment, the parent Fc-containing polypeptide is an Fc-fusion protein selected from the group consisting of: Arcalyst/rilonacept (IL1R-Fc fusion), Orencia/abatacept (CTLA-4-Fc fusion), Amevive/alefacept (LFA-3-Fc fusion), Anakinra-Fc fusion (IL-1Ra-Fc fusion protein), etanercept (TNFR-Fc fusion protein), FGF-21-Fc fusion protein, GLP-1-Fc fusion protein, RAGE-Fc fusion protein, ActRIIA-Fc fusion protein, ActRIIB-Fc fusion protein, glucagon-Fc fusion protein, oxyntomodulin-Fc-fusion protein, GM-CSF-Fc fusion protein, EPO-Fc fusion protein, Insulin-Fc fusion protein, proinsulin-Fc fusion protein and insulin precursor-Fc fusion protein, and analogs and variants thereof

### Methods of Treatment

The invention also comprises a method of treating a subject in need thereof comprising: administering to the subject a therapeutically effective amount of an Fc-containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region in the Fc-containing polypeptide, wherein the numbering is according to the EU index as in Kabat. In one embodiment, the Fc-containing polypeptide comprises N-glycans. In one embodiment, the Fc-containing polypeptide comprises sialylated N-glycans. The Fc-containing polypeptide of the invention can be administered by any route. In one embodiment, the Fc-containing polypeptide is administered parenterally. In one one embodiment, the Fc-containing polypeptide is administered subcutaneously.

In one embodiment, the subject has an inflammatory condition. In one embodiment, the method further comprises the administration of another anti-inflammatory compound. In another embodiment, the method further comprises the administration of an agent that increases expression of FcγRIIB. In another embodiment, the method further comprises the administration of an agent that binds DC-SIGN.

The term “inflammatory condition” refers to a condition that is characterized by abnormal or unwanted inflammation, such as autoimmune disease. Autoimmune diseases are disorders characterized by the chronic activation of immune cells under non-activating conditions. Examples include: psoriasis, inflammatory bowel diseases (e.g., Crohn's disease and ulcerative colitis), rheumatoid arthritis, psoriatic arthritis, multiple sclerosiss, systemic lupus erythematosus, type I diabetes, primary biliary cirrhosis, and transplant.

In one embodiment, the inflammatory condition is an autoimmune disease. In one embodiment, the inflammatory condition will be multiple sclerosis. In one embodiment, the inflammatory condition is systemic lupus erythematosus. In one embodiment, the inflammatory condition is type I diabetes.

In one embodiment, the inflammatory condition is a primary immunodeficiency syndrome, including congential agammaglobulinaemia and hypogammaglobulinaemia, common variable immunodeficiency, severed combined immunodeficiency, or Wiskott Aldrich syndrome.

In one embodiment, the inflammatory condition is a secondary immunodeficiency syndrome, including B-cell lymphocytic leukemia, HIV infection or an allogeneic bone marrow transplantation.

In one embodiment, the inflammatory condition is idiopathic thrombocytopenic purpura.

In one embodiment, the inflammatory condition is multiple myeloma.

In one embodiment, the inflammatory condition is Guillain-Barre syndrome.

In one embodiment, the inflammatory condition is Kawasaki disease.

In one embodiment, the inflammatory condition is chronic inflammatory demyelinating polyneropathy (CIDP).

In one embodiment, the inflammatory condition is autoimmune nuetropenia.

In one embodiment, the inflammatory condition is hemolytic anemia.

In one embodiment, the inflammatory condition is anti-Factor VIII autoimmune disease.

In one embodiment, the inflammatory condition is multifocal neuropathy.

In one embodiment, the inflammatory condition is systemic vasculitis (ANCA positive).

In one embodiment, the inflammatory condition is polymyositis.

In one embodiment, the inflammatory condition is dermatomyositis.

In one embodiment, the inflammatory condition is antiphospholipid syndrome.

In one embodiment, the inflammatory condition is sepsis syndrome.

In one embodiment, the inflammatory condition is graft-v-host disease.

In one embodiment, the inflammatory condition is allergy.

In one embodiment, the inflammatory condition is an anti-Rhesus factor D reaction.

In one embodiment, the inflammatory condition is an inflammatory condition of the cardiovascular system. The Fc-containing polypeptides of the invention may be used to treat atherosclerosis, atherothrombosis, coronary artery hypertension, acute coronary syndrome and heart failure, all of which are associated with inflammation.

In one embodiment, the inflammatory condition is an inflammatory condition of the central nervous system. In another embodiment, the inflammatory condition will be an inflammatory condition of the peripheral nervous system. For example, the Fc-containing polypeptides of the invention may be used for the treatment of, e.g., Alzheimer's disease, amyotrophic lateral sclerosis (a.k.a. ALS; Lou Gehrig's disease), ischemic brain injury, prion diseases, and HIV-associated dementia.

In one embodiment, the inflammatory condition is an inflammatory condition of the gastrointestinal tract. For example, the Fc-containing polypeptides of the invention may be used for treating inflammatory bowel disorders, e.g., Crohn's disease, ulcerative colitis, celiac disease, and irritable bowel syndrome.

In one embodiment, the inflammatory condition is psoriasis, atopic dermatitis, arthritis, including rheumatoid arthritis, osteoarthritis, and psoriatic arthritis.

In one embodiment, the inflammatory condition is steroid-dependent atopic dermatitis.

In one embodiment, the inflammatory condition is cachexia.

Examples of other inflammatory disorders that can be treated using the Fc-containing polypeptides of the invention also include: acne vulgaris, asthma, autoimmune diseases, chronic prostatitis, glomerulonephritis, hypersensitivities, pelvic inflammatory disease, reperfusion injury, sarcoidosis, transplant rejection, vasculitis, interstitial cystitis and myopathies.

A subject to be treated for an inflammatory condition can be identified by standard diagnosing techniques for the disorder. Optionally, the subject can be examined for the level or percentage of one or more of cytokines or cells a test sample obtained from the subject by methods known in the art. If the level or percentage is at or below a threshold value (which can be obtained from a normal subject), the subject is a candidate for treatment described herein. To confirm the inhibition or treatment, one can evaluate and/or verify the level or percentage of one or more of the above-mentioned cytokines or cells in the subject after treatment.

In another embodiment, the subject in need of treatment has infectious disease.

In another embodiment, the subject in need of treatment has cancer and the Fc-containing polypeptide is an immunomodulatory antibody that binds agonistically to co-stimulatory receptors or antigen-presenting cells and T cells to stimulate immunity (e.g. CD40, CD27, 41-BB, OX40, GITR, CD137 and HVEM). In embodiment, the invention comprises a method of treating a subject having cancer (or at risk of developing cancer) comprising: administering to the subject a therapeutically effective amount of an Fc-containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 and 439 of the Fc region in the Fc-containing polypeptide, wherein the numbering is according to the EU index as in Kabat, and wherein the Fc-containing polypeptide is an immunomodulatory antibody.

“Treating” or “treatment” refers to administration of a compound or agent to a subject who has a disorder with the purpose to cure, alleviate, relieve, remedy, delay the onset of, prevent, or ameliorate the disorder, the symptom of the disorder, the disease state secondary to the disorder, or the predisposition toward the disorder.

As used herein, the terms “therapeutically effective amount”, “therapeutically effective dose” and “effective amount” refer to an amount of an Fc-containing polypeptide of the invention that, when administered alone or in combination with an additional therapeutic agent to a cell, tissue, or subject, is effective to cause a measurable improvement in one or more symptoms of a disease or condition or the progression of such disease or condition. A therapeutically effective dose further refers to that amount of the Fc-containing polypeptide sufficient to result in at least partial amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of a therapeutic will result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity.

The agent can be administered in vivo or ex vivo, alone or co-administered in conjunction with other drugs or therapy, i.e., a cocktail therapy. As used herein, the term “co-administration” or “co-administered” refers to the administration of at least two agents or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

In an in vivo approach, a compound or agent is administered to a subject. Generally, the compound or agent is suspended in a pharmaceutically-acceptable carrier (such as, for example, but not limited to, physiological saline) and administered orally or by intravenous infusion, or injected or implanted subcutaneously, intramuscularly, intrathecally, intraperitoneally, intrarectally, intravaginally, intranasally, intragastrically, intratracheally, or intrapulmonarily.

The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.001-100 mg/kg.

In one embodiment, the Fc-containing polypeptide of the invention will be administered a dose of between 1 to 100 milligrams per kilograms of body weight. In one embodiment, the Fc-containing polypeptide of the invention will be administered a dose of between 0.001 to 10 milligrams per kilograms of body weight. In one embodiment, the Fc-containing polypeptide of the invention will be administered a dose of between 0.001 to 0.1 milligrams per kilograms of body weight. In one embodiment, the Fc-containing polypeptide of the invention will be administered a dose of between 0.001 to 0.01 milligrams per kilograms of body weight.

Fc-containing polypeptides can be provided by continuous infusion, or by doses administered, e.g., daily, 1-7 times per week, weekly, bi-weekly, monthly, bimonthly, quarterly, semiannually, annually etc.

### Pharmaceutical Formulations

The invention also comprises pharmaceutical formulations comprising an Fc-containing polypeptide of the invention and a pharmaceutically acceptable carrier. In one embodiment, the pharmaceutical formulation comprises and Fc-containing polypeptide comprising N-glycans. In one embodiment, the pharmaceutical formulation comprises and Fc-containing polypeptide comprising sialylated N-glycans.

In one embodiment, the Fc-containing polypeptide of the invention is an IgG antibody or antibody fragment. In one embodiment, the Fc-containing polypeptide of the invention is an IgG1 antibody or antibody fragment.

As utilized herein, the term “pharmaceutically acceptable” means a non-toxic material that does not interfere with the effectiveness of the biological activity of the active ingredient(s), approved by a regulatory agency of the Federal or a state government or listed in the U.S. Pharmacopoeia or other generally recognized pharmacopoeia for use in animals and, more particularly, in humans. The term “carrier” refers to a diluent, adjuvant, excipient, or vehicle with which the therapeutic is administered and includes, but is not limited to such sterile liquids as water and oils. The characteristics of the carrier will depend on the route of administration.

Pharmaceutical Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In certain embodiments, the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

In certain embodiments, the the Fc-containing polypeptides of the invention can be administered by an invasive route such as by injection (see above). In some embodiments of the invention, the Fc-containing polypeptides of the invention, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

Compositions can be administered with medical devices known in the art. For example, a pharmaceutical composition of the invention can be administered by injection with a hypodermic needle, including, e.g., a prefilled syringe or autoinjector.

The pharmaceutical compositions of the invention may also be administered with a needleless hypodermic injection device; such as the devices disclosed in U.S. Pat. Nos. 6,620,135; 6,096,002; 5,399,163; 5,383,851; 5,312,335; 5,064,413; 4,941,880; 4,790,824 or 4,596,556.

The pharmaceutical compositions of the invention may also be administered by infusion. Examples of well-known implants and modules form administering pharmaceutical compositions include: U.S. Pat. No. 4,487,603, which discloses an implantable micro-infusion pump for dispensing medication at a controlled rate; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments. Many other such implants, delivery systems, and modules are well known to those skilled in the art.

Alternately, one may administer the antibody in a local rather than systemic manner, for example, via injection of the antibody directly into an arthritic joint, often in a depot or sustained release formulation. Furthermore, one may administer the antibody in a targeted drug delivery system, for example, in a liposome coated with a tissue-specific antibody, targeting, for example, arthritic joint or pathogen-induced lesion characterized by immunopathology. The liposomes will be targeted to and taken up selectively by the afflicted tissue.

The administration regimen depends on several factors, including the serum or tissue turnover rate of the therapeutic antibody, the level of symptoms, the immunogenicity of the therapeutic antibody, and the accessibility of the target cells in the biological matrix. Preferably, the administration regimen delivers sufficient therapeutic antibody to effect improvement in the target disease state, while simultaneously minimizing undesired side effects. Accordingly, the amount of biologic delivered depends in part on the particular therapeutic antibody and the severity of the condition being treated. Guidance in selecting appropriate doses of therapeutic antibodies is available (see, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert, et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602).

### Example 1

### Construction of Library of Full-Length IgG1 Fc Variants in Pichia pastoris

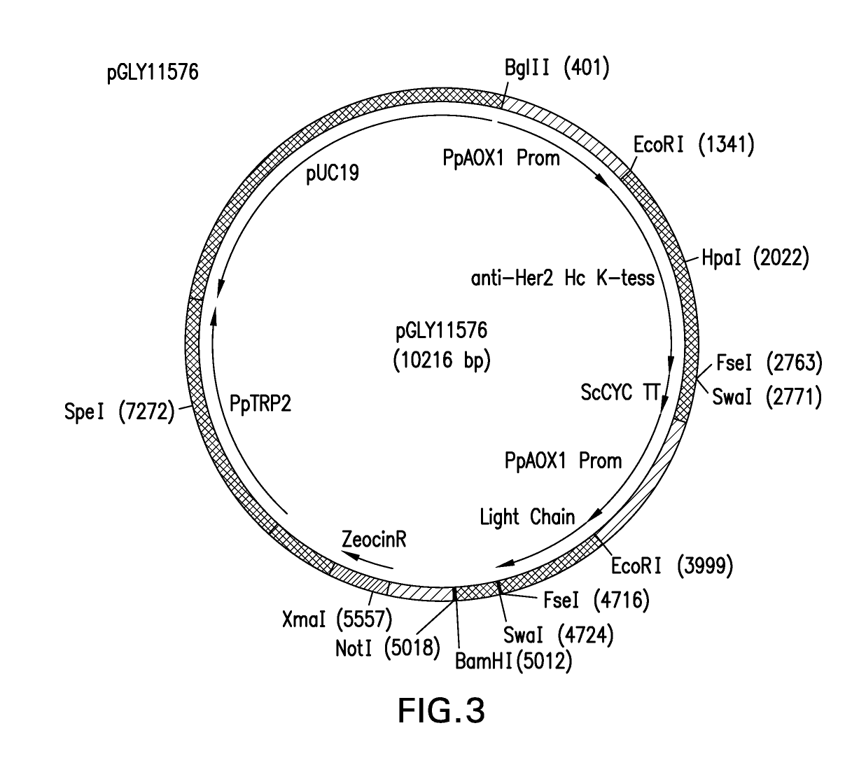

A library of Fc variants was constructed using error prone PCR to introduce random mutations in a region encompassing the CH2-CH3 domains of a human immunoglobulin G 1 (IgG1) gene. These PCR fragments encoding variant Fc molecules were cloned in frame to VH1-CH1 region of an anti-Her2 monoclonal antibody (mAb) under the expression of Pichia AOX1 promoter in pGLY11576 (FIG. 3). The complete amino acid sequence of the heavy chain of the Anti-Her2 antibody used in this experiment shown in SEQ ID NO:3. The VH1-CH1 sequence corresponds to amino acids 1-220 of SEQ ID NO: 3. Briefly, 1 microgram of ligation mixture was used to transform MegaX DH10B electro-competent E. coli cells (obtained from Life Technologies, Carlsbad, Calif.) by electroporation. Plated transformations resulted in 10⁷ clones carrying unique sequences encoding full-length IgG1 Fc variants. Clones were pooled cultured and lysed to isolate plasmid DNA using a Qiagen Maxi Plasmid Preparation kit (obtained from Qiagen, Germantown, Md.).

DNA isolated as described above (encoding full-length IgG1 Fc variants) was incubated for 2 hr with SpeI restriction enzyme at 37° C. Linearized plasmid DNA was precipitated with 3M sodium acetate pH 5.2 and isopropanol. Digested DNA was reconstituted in deionized water at concentration of 1 μg/μL. 1 mg of this DNA was introduced in a Pichia pastoris strain that is capable of producing sialylyated N-glycans and which contained plasmid pGLY11714 (FIG. 1, SEQ ID NO:4), generating a library of 10⁶ clones. These clones were scraped off plates and pooled in liquid media. The plasmid designated as pGLY11714 contains a cassette expressing IgG Light chain fused to surface anchor Sed1 (Lc-Sed1) on the cell surface. A random set of 80 yeast clones were cultured in a 96-well plate, induced and analyzed for secreting full length IgGs by SDS-PAGE. 65% of the clones were shown to produce full-length IgGs. (Pichia pastoris strains capable of producing sialylated N-glycans are described in the art, for example, in WO2011/149999.)

Construction of pGLY11714:

FIG. 1 contains a plasmid map of pGLY11714. Briefly, a polynucleotide encoding the N-terminus of a cell surface anchoring protein S. cerevisiae Sed1p that inherently contains an attached glycophosphotidylinositol (GPI) post-translational modification that anchors the protein on the yeast cell wall was linked to a nucleic acid sequence that encodes the human IgG2 anti PCSK9 (1F11) Light Chain (Lc). The plasmid pGLY11714 containing anti-PCSK9 Lc bait cassette was constructed using a codon optimized sequence of human IgG2 Lc (VL+CL) fragment, which was synthesized and fused in frame to the 3′ end of the nucleic acid sequence of S. cerevisiae α-mating factor signal sequence. The nucleic acid sequence encoding three repeats of the amino acids (GGGS) linker was used to link the nucleic acid sequence of Lc 3′ end to the 5′end of S. cerevisiae Sed1p. The construct was subcloned into pGLY9008 at EcoRI—SalI (replacing the Fc) by the contracting research organization (CRO) Genewiz (South Plainfield, N.J.). The resulting plasmid enables delivery of the Lc-SED1 cassette under the control of the Pichia pastoris AOX1 promoter at the URA6 locus in Pichia pastoris. SEQ ID NO: 4 provides the sequence of plasmid pGLY11714. SEQ ID NO:5 provides the sequence of the light chain of the anti-PCSK9 antibody. SEQ ID NO:6 provides the amino acid sequence alpha mating factor-antipPCSK9 Lc-(GGGS) linker-S. cerevisiae Sed1p.

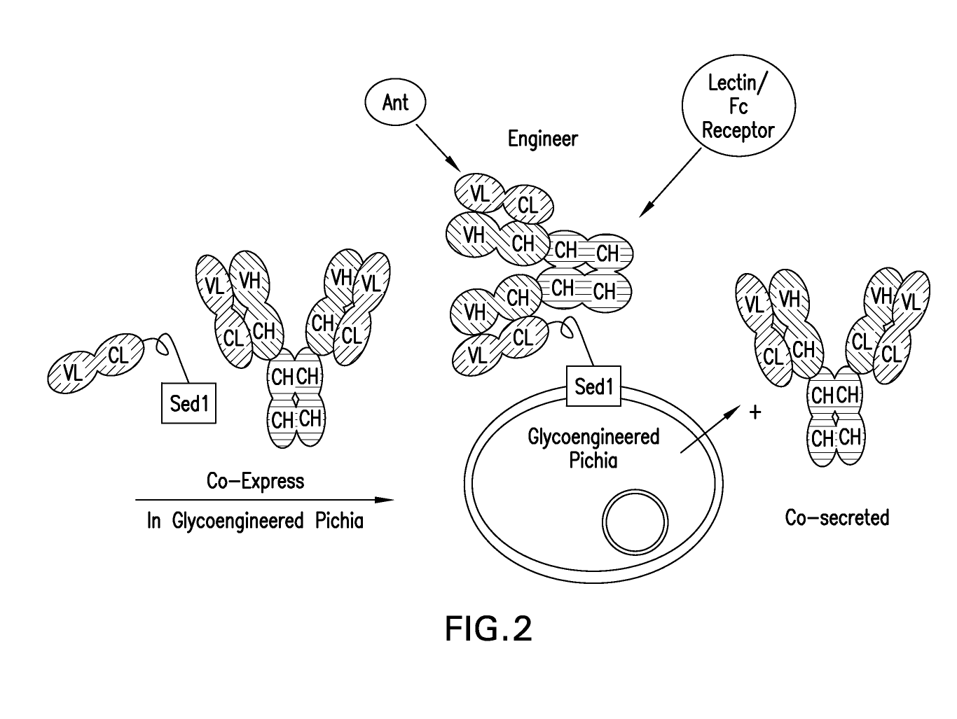

Strains that comprise plasmid pGLY11714 are able to display an antibody light chain and utilize the covalent interaction of this light chain with the heavy chain of an antibody molecule that is co-expressed in the same host. This interaction tethers the IgG molecule on the cell surface (See FIG. 2). This surface display allows for the discovery of novel Fc variants that possess specific desired biological properties, such as Fc receptor binding affinities. YGLY32444 also allows co-secretion of the displayed molecule allowing further in vitro analysis.

### Example 2

### Screening for Clones with Improved Binding to Human and Mouse FcγReceptor II-B (CD32-B) Using Biacore

Two independent approaches were followed to screen the yeast library described in Example 1 for Fc variants which display higher affinity to human and mouse FcγRIIB. One approach used Biacore (described herein in Example 2). Another approach (described in Example 3) used surface display.

Pichia clones generated from the full-length IgG1 library described in Example 1 were plated on solid medium and cultured individually in 96-well plates in BMGY. Log phase cultures were pelleted, resuspended, and incubated for 48 hr in liquid BMMY containing 2% methanol as carbon source to induce expression of AOX1 driven genes. Secreted IgG1 molecules were purified by passing culture supernatants through protein A coated beads and eluting bound IgGs at pH 3.0 into 96-well microtiter plates containing 1M Tris buffer (pH 8.0).

Purified IgGs were analyzed by SDS-PAGE for expression and assayed for binding to human or mouse FcγRIIB. The binding affinity of the IgG1 variants to human or mouse FcγRIIB was measured by surface plasmon resonance (SPR) on a Biacore T100 instrument with a carboxymethylated dextran chip (CM5) and 1×HBS-EP+(10 mM HEPES, 150 mM NaCl, 3 mM EDTA, and 0.05% Surfactant P20) as the running buffer. The CMS chip (GE Healthcare, Wauwatusa, Wis.) was immobilized on all flow cells with mouse anti-human IgG (Fc specific) (obtained from GE Healthcare, Wauwatusa, Wis.) according to the Biacore Human Antibody Capture Kit to ˜7000 RU. Purified full-length IgG1 variants containing mutations in Fc region (described in Example 1) were captured on the chip through immobilized mouse anti-human IgG (obtained from GE Healthcare, Wauwatusa, Wis.) followed by injection of human or mouse FcγRIIB. Human FcγRIIB was expressed in Chinese Hamster Ovary cells (Merck, Whithouse Station, N.J.) and mouse FcγRIIB was expressed in NS0 murine myeloma (R&D Systems, Minneapolis, Minn.)). Each flowcell was regenerated between each analyte injection with 3M MgCl for 40 s at 10 μl/min. Data was analyzed with Biacore T100 Evaluation Software using the 1/1 binding model for KD values, where reported. Amino acid compositions of novel variants were identified by DNA sequencing. Clones were tabulated based on response units (RU's) registered following receptor binding. These RU's were compared against wild-type IgG1 (weak binder) and IgG1 with point mutations S267E/L328F (Chu et al., Molecular Immunology, Volume 45, Issue 15, September 2008, Pages 3926-3933).

This assay identified the following Fc variants as exhibiting enhance binding to both human and mouse FcγRIIB receptors:
K317E/K326E/A339T/Q342R/K414R (clone 183-D4)
V284A/V305A/T307A/K360R
T250A/E272K/K360E/V422D/T437A
T223I/K320R/K322E
   The variants are identified by reference to the Kabat numbering system using wild type huma IgG1 as a reference and the single letter amino acid code. Thus, variant “T223I/K320R/K322E”, refers to an IgG1 variant that comprises: a mutation at position 223 that changes amino acid residue “T” to amino acid residue “I”; a mutation at position 320 that changes amino acid residue “K” to amino acid residue “R”; and a mutation at position 322 that changes amino acid residue “K” to amino acid residue “E”.

Table 1 shows the ratio of receptor binding response units (RU) to mAb capture response units (RU) compared to control variants. A higher ratio indicates higher degree of receptor binding.

TABLE 1

| | human FcgRIIB (RU)/ |
|---|---|
| Variant | mAb Capture (RU) |
| WT IgG1 (SEQ ID NO2) | 0.04 |
| F243A/V264A Fc (SEQ ID NO: 7) | 0.02 |
| F243A/V264A/S267E/L328F Fc (SEQ ID NO8) | 0.14 |
| V284A/V305A/T307A/K360R | 0.08 |
| T250A/E272K/K360E/V422D/T437A | 0.09 |
| T223I/K320R/K322E | 0.12 |

### Example 3

### Screening for Clones with Improved Binding to Human and Mouse FcγReceptor II-B (CD32-B) Using Surface Display

Pichia clones from the library described in Example 1 were cultured in a shake flask in growth medium BMGY for 24 hr and expression and surface display was initiated by resuspending and incubating for 24 hr in 2% methanol containing medium for at room temperature. After induction on methanol, 2 OD₆₀₀ of cells (˜10⁷ cells) were collected into a 1.5-ml microfuge tube and washed twice with phosphate-buffered saline (PBS) and then re-suspended in 100 μl of PBS containing 1 μl (2 μg) of fluorescently-labeled goat anti-human Fc DyeLight 647 (Jackson Immunoresearch Labs, West Grove, Pa.), and 3 μg of either of human or mouse FcγRIIB (human FcγRIIB was expressed in Chinese Hamster Ovary cells (Merck, Whithouse Station, N.J.) and mouse FcγRIIB was expressed in NS0 murine myeloma (R&D Systems, Minneapolis, Minn.)). The labeling cells/receptors mixtures were incubated at room temperature for 30 min. Receptors were detected with their respective fluorescently-labeled detection antibodies (purchased from R&D Systems, Minneapolis, Minn.). Cells were washed once with PBS and suspended in 300 μl of PBS for flow cytometric analysis and sorting. Clones with higher fluorescence intensity indicative of receptor labeling were isolated and cultured in liquid medium. 1000 clones were plated as single colonies on solid medium and analyzed by SPR as described in Example 2 to confirm FcγRIIB binding properties. The SPR analyses confirmed enrichment of IgG variants with enhanced mouse and human FcγRIIB binding. Amino acid compositions of novel variants were identified by DNA sequencing.

The variants identified according to this Example are as follows:
L309S/K340E/V412A
V302A/K326E/L328M/T335A/Y391H/K409R
K246R/A339T/K409R
K246E/T307A/K326E
N315S/I332T/N421D/S440P
K326E/N421D/K439E
K326E/I332T/I377V
   The variants are again identified by reference to the Kabat numbering system using wild type huma IgG1 as a reference and the single letter amino acid code. The results of the SPR analysis for the Fc variants identified according to this Example, and for for some previously identified variants, are shown in Table 2. The ratio of receptor binding response units (RU) to mAb capture response units (RU) was calculated and compared to control variants. A higher ratio indicates higher degree of receptor binding. In this Table, WT IgG1 refers to Herceptin (Genentech, San Fransico Calif.) Lot#724370.

TABLE 2

| | human | mouse |
|---|---|---|
| | FcγRIIB (RU)/ | FcγRIIB (RU)/ |
| Variant | mAb Capture (RU) | mAb Capture (RU) |
| WT IgG1 (Herceptin) | 0.04 | 0.01 |
| F243A/V264A | 0.01 | 0 |
| F243A/V264A/S267E/L328F | 0.14 | 0.03 |
| L309S/K340E/V412A | 0.22 | 0.19 |
| V302A/K326E/L328M/ | 0.22 | 0.25 |
| T335A/Y391H/K409R | | |
| K246R/A339T/K409R | 0.23 | 0.14 |
| K246E/T307A/K326E | 0.2 | 0.18 |
| N315S/I332T/N421D/S440P | 0.19 | 0.16 |
| K326E/N421D/K439E | 0.19 | 0.19 |
| K326E/I332T/I377V | 0.20 | 0.23 |

### Example 4

### Generation of Fc Fragments of Novel IgGs with Enhanced Binding to FcgRIIB

The sequences of novel binders isolated as described in Examples 2 and 3 were identified by DNA sequencing (Genewiz, South Plainfield, N.J.).

SEQ ID NO:7 refers to the amino acid sequence of a human IgG1 Fc region comprising mutations at amino acid positions 243 (F243A) and 264 (V264A), wherein the numbering is according to the EU system as in Kabat.

SEQ ID NO:8 refers to the amino acid sequence of a human IgG1 Fc region comprising mutations at amino acid positions 243 (F243A), 264 (V264A), 267 (S267E) and 328 (L238F), wherein the numbering is according to the EU system as in Kabat.

SEQ ID NO: 9 refers to the amino acid sequence of a human IgG1 Fc region comprising a mutation at amino acid position 241 (F241A), wherein the numbering is according to the EU system as in Kabat.

SEQ ID NO:10 is the amino acid sequence of a human IgG1 Fc region of a novel binder isolated as described in Example 2 (clone 183-D4), comprising mutations at Fc positions 317 (K317E), 326 (K326E), 339 (A339T), 342 (Q342R), and 414 (K414R), wherein the numbering is according to the EU system as in Kabat.

SEQ ID NO:11 is the amino acid sequence of a human IgG1 Fc region comprising mutations at Fc positions 317 (K317E), 326 (K326E) and 414 (K414R), wherein the numbering is according to the EU system as in Kabat.

SEQ ID NO:12 is the amino acid sequence of a human IgG1 Fc region comprising mutations at Fc positions 241 (F241A), 317 (K317E), 326 (K326E), 328 (L328F), 339 (A339T), 342 (Q342R) and 414 (K414R), wherein the numbering is according to the EU system as in Kabat.

SEQ ID NO:13 is the amino acid sequence of a human IgG1 Fc region comprising mutations at Fc positions 241 (F241A), 317 (K317E), 326 (K326E), 339 (A339T), 342 (Q342R) and 414 (K414R), wherein the numbering is according to the EU system as in Kabat.

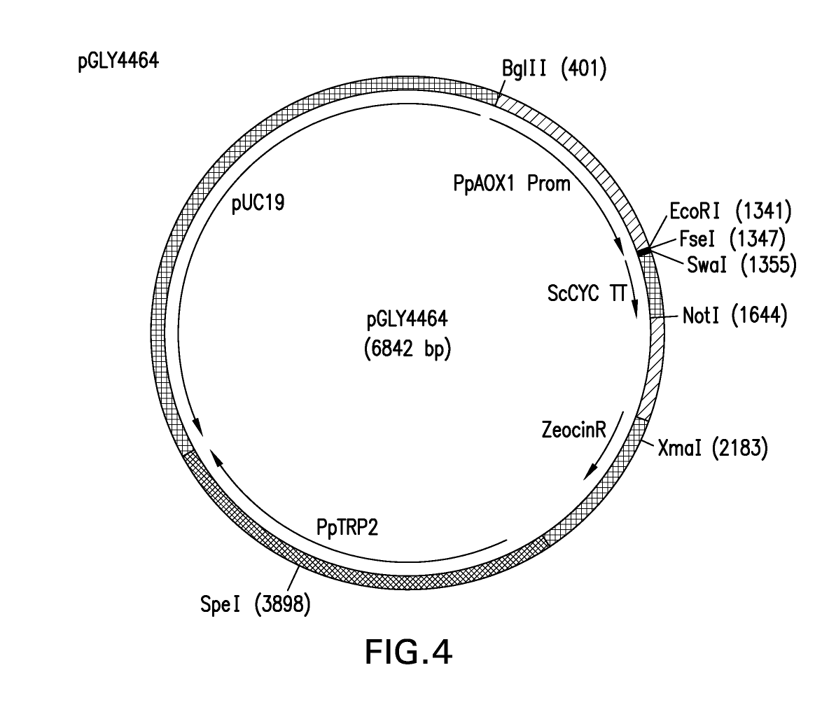

DNA regions encoding Fc fragments of SEQ ID NOs: 2 and 7-14 were synthesized by Genewiz (South Plainfield, N.J.) and cloned in Pichia expression vector pGLY4464 (FIG. 4) under the AOX1 promoter. The resulting plasmids contain Pichia pastoris TRP2 gene which is used to integrate expression cassettes into the yeast TRP2 locus.

Yeast strains producing each of these variant Fc fragments were generated by introducing SpeI linearized plasmids into humanized Pichia pastoris strain that have been engineered to produce sialylated N-glycans (of the type NANA₍₁₋₄₎Gal₍₁₋₄ GlcNAc₍₁₋₄₎₎Man₃GlcNAc₂). Resulting clones were fermented in 1 L bioreactor vessels to produce each of the described variants. Culture supernatants were passed over protein A coated beads to capture secreted Fc fragments. Purified Fc fragments were eluted at pH 3.0 into tubes containing 1M Tris (pH 8.0). Purified Fc fragments were analyzed by surface plasmon resonance (SPR) on Biacore for binding to human and mouse FcγRIIB as described in Example 2.

The results are shown in Table 3. “RU” or “Relative Response” refers to the measured change in mass on the surface of the sensor chip that is coated with equal moles of capture samples and exposed to equal amounts of the said receptors. 1 RU=1 pg/mm2, therefore an increase in response is related to an increase in mass on the surface due to binding of the receptor to the capture samples. Higher affinity capture samples display higher RUs. rhFcgRIIB (CHO) refers to recombinant human FcγRIIB expressed in CHO cells. rmFcgRIIB (NSO) refers to recombinant mouse FcγRIIB expressed in NSO cells. Table 4 lists fold change in rhFcgRIIb KD values of variants versus WT IgG1 (Herceptin).

TABLE 3

| | rhFcgRIIB | rmFcgRIIB |
|---|---|---|
| | Fold Increase in | Fold Increase in |
| Capture Sample | RU vs WT IgG1 | RU vs WT IgG1 |
| Herceptin (WT IgG1, Genentech, | 1.0 | 1.0 |
| San Fransico CA) Lot#724370 | | |
| F243A/V264A Fc (SEQ ID NO: 7) | 2.0 | 0.5 |
| F243A/V264A/S267E/L328F Fc | 7.0 | 2.4 |
| (SEQ ID NO8) | | |
| F241A Fc (SEQ ID NO: 9) | 2.7 | 2.3 |
| 183-D4 Fc (SEQ ID NO: 10) | 5.0 | 5.3 |
| 183-D4/F241A Fc (SEQ ID NO13) | 4.7 | 5.0 |
| K317E/K326E/K414R Fc (SEQ ID | 5.0 | 5.2 |
| NO11) | | |
| 183-D4/F241A/L328F Fc (SEQ ID | 1.2 | 1.3 |
| NO12) | | |

TABLE 4

| | Fold Improvement |
|---|---|
| | in KD vs WT |
| Sequence | IgG1 rhFcgRIIb |
| Herceptin (WT IgG1, Genentech, San Fransico | 1 |
| CA) Lot#724370 | |
| F243A/V264A/S267E/L328F Fc (SEQ ID NO8) | 11 |
| WT IgG1 Fc (SEQ ID NO2) | 0.8 |
| 183-D4 Fc (SEQ ID NO10) | 5 |

The results from this experiment confirm that the mutations identified in clone 183-D4 (corresponding to mutations at Fc positions 317 (K317E), 326 (K326E), 339 (A339T), 342 (Q342R), and 414 (K414R)) result in enhanced binding to both human and mouse FcγRIIB. These results were still observed in a mutant that only contained the mutations at positions 317, 326 and 414. The addition of a mutation at position 241 did not have an impact on binding to human and mouse FcγRIIB.

SEQUENCE LISTING

| SEQ ID | | |
|---|---|---|
| NO: | DESCRIPTION | SEQUENCE |
| 1 | Human IgG1 Fc | T C P P C P A P E L L G G P S V F L F P P |
| | region | K P K D T L M I S R T P E V T C V V V D V |
| | (wildtype) | S H E D P E V K F N W Y V D G V E V H N A |
| | | K T K P R E E Q Y N S T Y R V V S V L T V |
| | | L H Q D W L N G K E Y K C K V S N K A L P |
| | | A P I E K T I S K A K G Q P R E P Q V Y T |
| | | L P P S R D E L T K N Q V S L T C L V K G |
| | | F Y P S D I A V E W E S N G Q P E N N Y K |
| | | T T P P V L D S D G S F F L Y S K L T V D |
| | | K S R W Q Q G N V F S C S V M H E A L H N |
| | | H Y T Q K S L S L S P G |
| 2 | Human IgG1 Fc | A E P K S C D K T H T C P P C P A P E L L |
| | region | G G P S V F L F P P K P K D T L M I S R T |
| | (wildtype) | P E V T C V V V D V S H E D P E V K F N W |
| | | Y V D G V E V H N A K T K P R E E Q Y N S |
| | | T Y R V V S V L T V L H Q D W L N G K E Y |
| | | K C K V S N K A L P A P I E K T I S K A K |
| | | G Q P R E P Q V Y T L P P S R D E L T K N |
| | | Q V S L T C L V K G F Y P S D I A V E W E |
| | | S N G Q P E N N Y K T T P P V L D S D G S |
| | | F F L Y S K L T V D K S R W Q Q G N V F S |
| | | C S V M H E A L H N H Y T Q K S L S L S P |
| | | G |
| 3 | Heavy chain | E V Q L V E S G G G L V Q P G G S L R L S |
| | amino acid | C A A S G F N I K D T Y I H W V R Q A P G |
| | sequence of Her2 | K G L E W V A R I Y P T N G Y T R Y A D S |
| | IgG1 antibody | V K G R F T I S A D T S K N T A Y L Q M N |
| | | S L R A E D T A V Y Y C S R W G G D G F Y |
| | | A M D Y W G Q G T L V T V S S A S T K G P |
| | | S V F P L A P S S K S T S G G T A A L G C |
| | | L V K D Y F P E P V T V S W N S G A L T S |
| | | G V H T F P A V L Q S S G L Y S L S S V V |
| | | T V P S S S L G T Q T Y I C N V N H K P S |
| | | N T K V D K K V E P K S C D K T H T C P P |
| | | C P A P E L L G G P S V F L F P P L P L D |
| | | T L M I S R T P E V T C V V V D V S H E D |
| | | P E V K F N W Y V D G V E V H N A K T K P |
| | | R E E Q Y N S T Y R V V S V L T V L H Q D |
| | | W L N G K E Y K C K V S N K A L P A P I E |
| | | K T I S K A K G Q P R E P Q V Y T L P P S |
| | | R E E M T K N Q V S L T C L V K G F Y P S |
| | | D I A V E W E S N G Q P E N N Y K T T P P |
| | | V L D S D G S F F L Y S K L T V D K S R W |
| | | Q Q G N V F S C S V M H E A L H N H Y T Q |
| | | K S L S L S P G |
| 4 | Nucleic Acid | TCGCGCGTTTCGGTGATGACGGTGAAAACCTCTGACACATGCAGCTCCCGGAGACGGTCACA |
| | Sequence of | GCTTGTCTGTAAGCGGATGCCGGGAGCAGACAAGCCCGTCAGGGCGCGTCAGCGGGTGTTGG |
| | pGLY11714 | CGGGTGTCGGGGCTGGCTTAACTATGCGGCATCAGAGCAGATTGTACTGAGAGTGCACCATA |
| | | TGCGGTGTGAAATACCGCACAGATGCGTAAGGAGAAAATACCGCATCAGGCGCCATTCGCCA |
| | | TTCAGGCTGCGCAACTGTTGGGAAGGGCGATCGGTGCGGGCCTCTTCGCTATTACGCCAGCT |
| | | GGCGAAAGGGGGATGTGCTGCAAGGCGATTAAGTTGGGTAACGCCAGGGTTTTCCCAGTCAC |
| | | GACGTTGTAAAACGACGGCCAGTGAATTGAGATCTAACATCCAAAGACGAAAGGTTGAATGA |
| | | AACCTTTTTGCCATCCGACATCCACAGGTCCATTCTCACACATAAGTGCCAAACGCAACAGG |
| | | AGGGGATACACTAGCAGCAGACCGTTGCAAACGCAGGACCTCCACTCCTCTTCTCCTCAACA |
| | | CCCACTTTTGCCATCGAAAAACCAGCCCAGTTATTGGGCTTGATTGGAGCTCGCTCATTCCA |
| | | ATTCCTTCTATTAGGCTACTAACACCATGACTTTATTAGCCTGTCTATCCTGGCCCCCCTGG |
| | | CGAGGTTCATGTTTGTTTATTTCCGAATGCAACAAGCTCCGCATTACACCCGAACATCACTC |
| | | CAGATGAGGGCTTTCTGAGTGTGGGGTCAAATAGTTTCATGTTCCCCAAATGGCCCAAAACT |
| | | GACAGTTTAAACGCTGTCTTGGAACCTAATATGACAAAAGCGTGATCTCATCCAAGATGAAC |
| | | TAAGTTTGGTTCGTTGAAATGCTAACGGCCAGTTGGTCAAAAAGAAACTTCCAAAAGTCGGC |
| | | ATACCGTTTGTCTTGTTTGGTATTGATTGACGAATGCTCAAAAATAATCTCATTAATGCTTA |
| | | GCGCAGTCTCTCTATCGCTTCTGAACCCCGGTGCACCTGTGCCGAAACGCAAATGGGGAAAC |
| | | ACCCGCTTTTTGGATGATTATGCATTGTCTCCACATTGTATGCTTCCAAGATTCTGGTGGGA |
| | | ATACTGCTGATAGCCTAACGTTCATGATCAAAATTTAACTGTTCTAACCCCTACTTGACAGC |
| | | AATATATAAACAGAAGGAAGCTGCCCTGTCTTAAACCTTTTTTTTTATCATCATTATTAGCT |
| | | TACTTTCATAATTGCGACTGGTTCCAATTGACAAGCTTTTGATTTTAACGACTTTTAACGAC |
| | | AACTTGAGAAGATCAAAAAACAACTAATTATTCGAAACGGAATTCACGATGAGATTCCCATC |
| | | CATCTTCACTGCTGTTTTGTTCGCTGCTTCCTCTGCTTTGGCTGACATTCAAATGACTCAGT |
| | | CCCCATCTTCCTTGTCTGCTTCCGTTGGTGACAGAGTTACTATCACTTGTAAGGCTTCCCAG |
| | | AACGTTGGAACTAACGTTGTTTGGTATCAGCAGAAGCCAGGTAAGGCTCCAAAGGCTTTGAT |
| | | TCACTCCGCTTCATACAGATACTCCGGTGTTCCATCCAGATTCTCTGGTTCTGGTTCCGGTA |
| | | CTGACTTTACTTTGACTATCTCCTCATTGCAGCCAGAGGACTTCGCTACTTACTACTGTCAG |
| | | CAGTACAAGACTTACCCATACACTTTCGGTCAGGGTACCAAGGTTGAGATCAAGAGAACTGT |
| | | TGCTGCTCCATCCGTTTTCATTTTCCCACCATCCGACGAACAGTTGAAGTCTGGTACAGCTT |
| | | CCGTTGTTTGTTTGTTGAACAACTTCTACCCAAGAGAGGCTAAGGTTCAGTGGAAGGTTGAC |
| | | AACGCTTTGCAATCCGGTAACTCCCAAGAATCCGTTACTGAGCAAGACTCTAAGGACTCCAC |
| | | TTACTCCTTGTCCTCCACTTTGACTTTGTCCAAGGCTGATTACGAGAAGCACAAGGTTTACG |
| | | CTTGTGAGGTTACACATCAGGGTTTGTCCTCCCCAGTTACTAAGTCCTTCAACAGAGGAGAG |
| | | TGTGGTGGTGGTGGTTCCGGTGGTGGTGGTTCTGGTGGTGGTGGTTCTGTCGACCAATTCTC |
| | | TAACTCTACTTCCGCTTCCTCTACTGACGTTACTTCCTCCTCCTCTATTTCTACTTCCTCCG |
| | | GTTCCGTTACTATTACTTCCTCTGAGGCTCCAGAATCTGACAACGGTACTTCTACTGCTGCT |
| | | CCAACTGAAACTTCTACTGAGGCTCCTACTACTGCTATTCCAACTAACGGAACTTCCACAGA |
| | | GGCTCCAACAACAGCTATCCCTACAAACGGTACATCCACTGAAGCTCCTACTGACACTACTA |
| | | CAGAAGCTCCAACTACTGCTTTGCCTACTAATGGTACATCAACAGAGGCTCCTACAGATACA |
| | | ACAACTGAAGCTCCAACAACTGGATTGCCAACAAACGGTACTACTTCTGCTTTCCCACCAAC |
| | | TACTTCCTTGCCACCATCCAACACTACTACTACTCCACCATACAACCCATCCACTGACTACA |
| | | CTACTGACTACACAGTTGTTACTGAGTACACTACTTACTGTCCAGAGCCAACTACTTTCACA |
| | | ACAAACGGAAAGACTTACACTGTTACTGAGCCTACTACTTTGACTATCACTGACTGTCCATG |
| | | TACTATCGAGAAGCCAACTACTACTTCCACTACAGAGTATACTGTTGTTACAGAATACACAA |
| | | CATATTGTCCTGAGCCAACAACATTCACTACTAATGGAAAAACATACACAGTTACAGAACCA |
| | | ACTACATTGACAATTACAGATTGTCCTTGTACAATTGAGAAGTCCGAGGCTCCTGAATCTTC |
| | | TGTTCCAGTTACTGAATCCAAGGGTACTACTACTAAAGAAACTGGTGTTACTACTAAGCAGA |
| | | CTACTGCTAACCCATCCTTGACTGTTTCCACTGTTGTTCCAGTTTCTTCCTCTGCTTCTTCC |
| | | CACTCCGTTGTTATCAACTCCAACGGTGCTAACGTTGTTGTTCCTGGTGCTTTGGGATTGGC |
| | | TGGTGTTGCTATGTTGTTCTTGTAATAGGGCCGGCCATTTAAATACAGGCCCCTTTTCCTTT |
| | | GTCGATATCATGTAATTAGTTATGTCACGCTTACATTCACGCCCTCCTCCCACATCCGCTCT |
| | | AACCGAAAAGGAAGGAGTTAGACAACCTGAAGTCTAGGTCCCTATTTATTTTTTTTAATAGT |
| | | TATGTTAGTATTAAGAACGTTATTTATATTTCAAATTTTTCTTTTTTTTCTGTACAAACGCG |
| | | TGTACGCATGTAACATTATACTGAAAACCTTGCTTGAGAAGGTTTTGGGACGCTCGAAGGCT |
| | | TTAATTTGCAAGCTGGATCCGCGGCCGCTTACGCGCCGTTCTTCGCTTGGTCTTGTATCTCC |
| | | TTACACTGTATCTTCCCATTTGCGTTTAGGTGGTTATCAAAAACTAAAAGGAAAAATTTCAG |
| | | ATGTTTATCTCTAAGGTTTTTTCTTTTTACAGTATAACACGTGATGCGTCACGTGGTACTAG |
| | | ATTACGTAAGTTATTTTGGTCCGGTGGGTAAGTGGGTAAGAATAGAAAGCATGAAGGTTTAC |
| | | AAAAACGCAGTCACGAATTATTGCTACTTCGAGCTTGGAACCACCCCAAAGATTATATTGTA |
| | | CTGATGCACTACCTTCTCGATTTTGCTCCTCCAAGAACCTACGAAAAACATTTCTTGAGCCT |
| | | TTTCAACCTAGACTACACATCAAGTTATTTAAGGTATGTTCCGTTAACATGTAAGAAAAGGA |
| | | GAGGATAGATCGTTTATGGGGTACGTCGCCTGATTCAAGCGTGACCATTCGAAGAATAGGCC |
| | | TTCGAAAGCTGAATAAAGCAAATGTCAGTTGCGATTGGTATGCTGACAAATTAGCATAAAAA |
| | | GCAATAGACTTTCTAACCACCTGTTTTTTTCCTTTTACTTTATTTATATTTTGCCACCGTAC |
| | | TAACAAGTTCAGACAAATTAATTAACACCATGTCAGAAGATCAAAAAAGTGAAAATTCCGTA |
| | | CCTTCTAAGGTTAATATGGTGAATCGCACCGATATACTGACTACGATCAAGTCATTGTCATG |
| | | GCTTGACTTGATGTTGCCATTTACTATAATTCTCTCCATAATCATTGCAGTAATAATTTCTG |
| | | TCTATGTGCCTTCTTCCCGTCACACTTTTGACGCTGAAGGTCATCCCAATCTAATGGGAGTG |
| | | TCCATTCCTTTGACTGTTGGTATGATTGTAATGATGATTCCCCCGATCTGCAAAGTTTCCTG |
| | | GGAGTCTATTCACAAGTACTTCTACAGGAGCTATATAAGGAAGCAACTAGCCCTCTCGTTAT |
| | | TTTTGAATTGGGTCATCGGTCCTTTGTTGATGACAGCATTGGCGTGGATGGCGCTATTCGAT |
| | | TATAAGGAATACCGTCAAGGCATTATTATGATCGGAGTAGCTAGATGCATTGCCATGGTGCT |
| | | AATTTGGAATCAGATTGCTGGAGGAGACAATGATCTCTGCGTCGTGCTTGTTATTACAAACT |
| | | CGCTTTTACAGATGGTATTATATGCACCATTGCAGATATTTTACTGTTATGTTATTTCTCAT |
| | | GACCACCTGAATACTTCAAATAGGGTATTATTCGAAGAGGTTGCAAAGTCTGTCGGAGTTTT |
| | | TCTCGGCATACCACTGGGAATTGGCATTATCATACGTTTGGGAAGTCTTACCATAGCTGGTA |
| | | AAAGTAATTATGAAAAATACATTTTGAGATTTATTTCTCCATGGGCAATGATCGGATTTCAT |
| | | TACACTTTATTTGTTATTTTTATTAGTAGAGGTTATCAATTTATCCACGAAATTGGTTCTGC |
| | | AATATTGTGCTTTGTCCCATTGGTGCTTTACTTCTTTATTGCATGGTTTTTGACCTTCGCAT |
| | | TAATGAGGTACTTATCAATATCTAGGAGTGATACACAAAGAGAATGTAGCTGTGACCAAGAA |
| | | CTACTTTTAAAGAGGGTCTGGGGAAGAAAGTCTTGTGAAGCTAGCTTTTCTATTACGATGAC |
| | | GCAATGTTTCACTATGGCTTCAAATAATTTTGAACTATCCCTGGCAATTGCTATTTCCTTAT |
| | | ATGGTAACAATAGCAAGCAAGCAATAGCTGCAACATTTGGGCCGTTGCTAGAAGTTCCAATT |
| | | TTATTGATTTTGGCAATAGTCGCGAGAATCCTTAAACCATATTATATATGGAACAATAGAAA |
| | | TTAATTAACAGGCCCCTTTTCCTTTGTCGATATCATGTAATTAGTTATGTCACGCTTACATT |
| | | CACGCCCTCCTCCCACATCCGCTCTAACCGAAAAGGAAGGAGTTAGACAACCTGAAGTCTAG |
| | | GTCCCTATTTATTTTTTTTAATAGTTATGTTAGTATTAAGAACGTTATTTATATTTCAAATT |
| | | TTTCTTTTTTTTCTGTACAAACGCGTGTACGCATGTAACATTATACTGAAAACCTTGCTTGA |
| | | GAAGGTTTTGGGACGCTCGAAGGCTTTAATTTGCAAGCTGCGGCCTAAGGCGCGCCAGGCCA |
| | | TAATGGCCCAAATGCAAGAGGACATTAGAAATGTGTTTGGTAAGAACATGAAGCCGGAGGCA |
| | | TACAAACGATTCACAGATTTGAAGGAGGAAAACAAACTGCATCCACCGGAAGTGCCAGCAGC |
| | | CGTGTATGCCAACCTTGCTCTCAAAGGCATTCCTACGGATCTGAGTGGGAAATATCTGAGAT |
| | | TCACAGACCCACTATTGGAACAGTACCAAACCTAGTTTGGCCGATCCATGATTATGTAATGC |
| | | ATATAGTTTTTGTCGATGCTCACCCGTTTCGAGTCTGTCTCGTATCGTCTTACGTATAAGTT |
| | | CAAGCATGTTTACCAGGTCTGTTAGAAACTCCTTTGTGAGGGCAGGACCTATTCGTCTCGGT |
| | | CCCGTTGTTTCTAAGAGACTGTACAGCCAAGCGCAGAATGGTGGCATTAACCATAAGAGGAT |
| | | TCTGATCGGACTTGGTCTATTGGCTATTGGAACCACCCTTTACGGGACAACCAACCCTACCA |
| | | AGACTCCTATTGCATTTGTGGAACCAGCCACGGAAAGAGCGTTTAAGGACGGAGACGTCTCT |
| | | GTGATTTTTGTTCTCGGAGGTCCAGGAGCTGGAAAAGGTACCCAATGTGCCAAACTAGTGAG |
| | | TAATTACGGATTTGTTCACCTGTCAGCTGGAGACTTGTTACGTGCAGAACAGAAGAGGGAGG |
| | | GGTCTAAGTATGGAGAGATGATTTCCCAGTATATCAGAGATGGACTGATAGTACCTCAAGAG |
| | | GTCACCATTGCGCTCTTGGAGCAGGCCATGAAGGAAAACTTCGAGAAAGGGAAGACACGGTT |
| | | CTTGATTGATGGATTCCCTCGTAAGATGGACCAGGCCAAAACTTTTGAGGAAAAAGTCGCAA |
| | | AGTCCAAGGTGACACTTTTCTTTGATTGTCCCGAATCAGTGCTCCTTGAGAGATTACTTAAA |
| | | AGAGGACAGACAAGCGGAAGAGAGGATGATAATGCGGAGAGTATCAAAAAAAGATTCAAAAC |
| | | ATTCGTGGAAACTTCGATGCCTGTGGTGGACTATTTCGGGAAGCAAGGACGCGTTTTGAAGG |
| | | TATCTTGTGACCACCCTGTGGATCAAGTGTATTCACAGGTTGTGTCGGTGCTAAAAGAGAAG |
| | | GGGATCTTTGCCGATAACGAGACGGAGAATAAATAAACATTGTAATAAGATTTAGACTGTGA |
| | | ATGTTCTATGTAATATTTTTCGAGATACTGTATCTATCTGGTGTACCGTATCACTCTGGACT |
| | | TGCAAACTCATTGATTACTTGTGCAATGGGCAAGAAGGATAGCTCTAGAAAGAAGAAGAAAA |
| | | AGGAGCCGCCTGAAGAGCTGGATCTTTCCGAGGTTGTTCCAACTTTTGGTTATGAGGAATTT |
| | | CATGTTGAGCAAGAGGAGAATCCGGTCGATCAAGACGAACTTGACGGCCATAATGGCCTAGC |
| | | TTGGCGTAATCATGGTCATAGCTGTTTCCTGTGTGAAATTGTTATCCGCTCACAATTCCACA |
| | | CAACATACGAGCCGGAAGCATAAAGTGTAAAGCCTGGGGTGCCTAATGAGTGAGCTAACTCA |
| | | CATTAATTGCGTTGCGCTCACTGCCCGCTTTCCAGTCGGGAAACCTGTCGTGCCAGCTGCAT |
| | | TAATGAATCGGCCAACGCGCGGGGAGAGGCGGTTTGCGTATTGGGCGCTCTTCCGCTTCCTC |
| | | GCTCACTGACTCGCTGCGCTCGGTCGTTCGGCTGCGGCGAGCGGTATCAGCTCACTCAAAGG |
| | | CGGTAATACGGTTATCCACAGAATCAGGGGATAACGCAGGAAAGAACATGTGAGCAAAAGGC |
| | | CAGCAAAAGGCCAGGAACCGTAAAAAGGCCGCGTTGCTGGCGTTTTTCCATAGGCTCCGCCC |
| | | CCCTGACGAGCATCACAAAAATCGACGCTCAAGTCAGAGGTGGCGAAACCCGACAGGACTAT |
| | | AAAGATACCAGGCGTTTCCCCCTGGAAGCTCCCTCGTGCGCTCTCCTGTTCCGACCCTGCCG |
| | | CTTACCGGATACCTGTCCGCCTTTCTCCCTTCGGGAAGCGTGGCGCTTTCTCATAGCTCACG |
| | | CTGTAGGTATCTCAGTTCGGTGTAGGTCGTTCGCTCCAAGCTGGGCTGTGTGCACGAACCCC |
| | | CCGTTCAGCCCGACCGCTGCGCCTTATCCGGTAACTATCGTCTTGAGTCCAACCCGGTAAGA |
| | | CACGACTTATCGCCACTGGCAGCAGCCACTGGTAACAGGATTAGCAGAGCGAGGTATGTAGG |
| | | CGGTGCTACAGAGTTCTTGAAGTGGTGGCCTAACTACGGCTACACTAGAAGGACAGTATTTG |
| | | GTATCTGCGCTCTGCTGAAGCCAGTTACCTTCGGAAAAAGAGTTGGTAGCTCTTGATCCGGC |
| | | AAACAAACCACCGCTGGTAGCGGTGGTTTTTTTGTTTGCAAGCAGCAGATTACGCGCAGAAA |
| | | AAAAGGATCTCAAGAAGATCCTTTGATCTTTTCTACGGGGTCTGACGCTCAGTGGAACGAAA |
| | | ACTCACGTTAAGGGATTTTGGTCATGAGATTATCAAAAAGGATCTTCACCTAGATCCTTTTA |
| | | AATTAAAAATGAAGTTTTAAATCAATCTAAAGTATATATGAGTAAACTTGGTCTGACAGTTA |
| | | CCAATGCTTAATCAGTGAGGCACCTATCTCAGCGATCTGTCTATTTCGTTCATCCATAGTTG |
| | | CCTGACTCCCCGTCGTGTAGATAACTACGATACGGGAGGGCTTACCATCTGGCCCCAGTGCT |
| | | GCAATGATACCGCGAGACCCACGCTCACCGGCTCCAGATTTATCAGCAATAAACCAGCCAGC |
| | | CGGAAGGGCCGAGCGCAGAAGTGGTCCTGCAACTTTATCCGCCTCCATCCAGTCTATTAATT |
| | | GTTGCCGGGAAGCTAGAGTAAGTAGTTCGCCAGTTAATAGTTTGCGCAACGTTGTTGCCATT |
| | | GCTACAGGCATCGTGGTGTCACGCTCGTCGTTTGGTATGGCTTCATTCAGCTCCGGTTCCCA |
| | | ACGATCAAGGCGAGTTACATGATCCCCCATGTTGTGCAAAAAAGCGGTTAGCTCCTTCGGTC |
| | | CTCCGATCGTTGTCAGAAGTAAGTTGGCCGCAGTGTTATCACTCATGGTTATGGCAGCACTG |
| | | CATAATTCTCTTACTGTCATGCCATCCGTAAGATGCTTTTCTGTGACTGGTGAGTACTCAAC |
| | | CAAGTCATTCTGAGAATAGTGTATGCGGCGACCGAGTTGCTCTTGCCCGGCGTCAATACGGG |
| | | ATAATACCGCGCCACATAGCAGAACTTTAAAAGTGCTCATCATTGGAAAACGTTCTTCGGGG |
| | | CGAAAACTCTCAAGGATCTTACCGCTGTTGAGATCCAGTTCGATGTAACCCACTCGTGCACC |
| | | CAACTGATCTTCAGCATCTTTTACTTTCACCAGCGTTTCTGGGTGAGCAAAAACAGGAAGGC |
| | | AAAATGCCGCAAAAAAGGGAATAAGGGCGACACGGAAATGTTGAATACTCATACTCTTCCTT |
| | | TTTCAATATTATTGAAGCATTTATCAGGGTTATTGTCTCATGAGCGGATACATATTTGAATG |
| | | TATTTAGAAAAATAAACAAATAGGGGTTCCGCGCACATTTCCCCGAAAAGTGCCACCTGACG |
| | | TCTAAGAAACCATTATTATCATGACATTAACCTATAAAAATAGGCGTATCACGAGGCCCTTT |
| | | CGTC |
| 5 | Amino acid | DIQMTQSPSSLSASVGDRVTITCKASQNVGTNVVWYQQKPGKAPKALIHSASYRYSGVPSRF |
| | sequence of | SGSGSGTDFTLTISSLQPEDFATYYCQQYKTYPYTFGQGTKVEIKRTVAAPSVFIFPPSDEQ |
| | anti-PCSK9 Light | LKSGTASVVCLLNNFYPREAKVQWKVDNALQSGNSQESVTEQDSKDSTYSLSSTLTLSKADY |
| | Chain | EKHKVYACEVTHQGLSSPVTKSFNRGEC |
| 6 | Amino acid | MRFPSIFTAVLFAASSALADIQMTQSPSSLSASVGDRVTITCKASQNVGTNVVWYQQKPGKA |
| | sequence of | PKALIHSASYRYSGVPSRFSGSGSGTDFTLTISSLOPEDFATYYCOOYKTYPYTFGOGTKVE |
| | alpha mating | IKRTVAAPSVFIFPPSDEOLKSGTASVVCLLNNFYPREAKVOWKVDNALOSGNSOESVTEOD |
| | factor- | SKDSTYSLSSTLTLSKADYEKHKVYACEVTHQGLSSPVTKSFNRGECGGGGSGGGGSGGGGS |
| | antipPCSK9 Lc- | VDQFSNSTSASSTDVTSSSSISTSSGSVTITSSEAPESDNGTSTAAPTETSTEAPTTAIPTN |
| | (GGGS, SEQ ID | GTSTEAPTTAIPTNGTSTEAPTDTTTEAPTTALPTNGTSTEAPTDTTTEAPTTGLPTNGTTS |
| | NO: 3) linker- | AFPPTTSLPPSNTTTTPPYNPSTDYTTDYTVVTEYTTYCPEPTTFTTNGKTYTVTEPTTLTI |
| | S. cerevisiae | TDCPCTIEKPTTTSTTEYTVVTEYTTYCPEPTTFTTNGKTYTVTEPTTLTITDCPCTIEKSE |
| | Sed1p | APESSVPVTESKGTTTKETGVTTKQTTANPSLTVSTVVPVSSSASSHSVVINSNGANVVVPG |
| | | ALGLAGVAMLFL |
| 7 | Amino acid | AEPKSCDKTHTCPPCPAPELLGGPSVFLAPPKPKDTLMISRTPEVTCVVADVSHEDPEVKFN |
| | sequence of | WYVDGVEVHNAKTKPREEQYNSTYRVVSVLTVLHQDWLNGKEYKCKVSNKALPAPIEKTISK |
| | human IgG1 Fc | AKGQPREPQVYTLPPSREEMTKNQVSLTCLVKGFYPSDIAVEWESNGQPENNYKTTPPVLDS |
| | polypeptide | DGSFFLYSKLTVDKSRWQQGNVFSCSVMHEALHNHYTQKSLSLSPG |
| | comprising | |
| | mutations at | |
| | positions F243A | |
| | and V264A | |
| 8 | Amino acid | AEPKSCDKTHTCPPCPAPELLGGPSVFLAPPKPKDTLMISRTPEVTCVVADVEHEDPEVKFN |
| | sequence of | WYVDGVEVHNAKTKPREEQYNSTYRVVSVLTVLHQDWLNGKEYKCKVSNKAFPAPIEKTISK |
| | human IgG1 Fc | AKGQPREPQVYTLPPSREEMTKNQVSLTCLVKGFYPSDIAVEWESNGQPENNYKTTPPVLDS |
| | polypeptide | DGSFFLYSKLTVDKSRWQQGNVFSCSVMHEALHNHYTQKSLSLSPG |
| | comprising | |
| | mutations at | |
| | positions 243 | |
| | (F243A), 264 | |
| | (V264A), 267 | |
| | (S267E) and 328 | |
| | (L238F), | |
| 9 | Amino acid | AEPKSCDKTHTCPPCPAPELLGGPSVALFPPKPKDTLMISRTPEVTCVVVDVSHEDPEVKFN |
| | sequence of | WYVDGVEVHNAKTKPREEQYNSTYRVVSVLTVLHQDWLNGKEYKCKVSNKALPAPIEKTISK |
| | human IgG1 Fc | AKGQPREPQVYTLPPSREEMTKNQVSLTCLVKGFYPSDIAVEWESNGQPENNYKTTPPVLDS |
| | polypeptide | DGSFFLYSKLTVDKSRWQQGNVFSCSVMHEALHNHYTQKSLSLSPG |
| | comprising a | |
| | mutation at | |
| | position 241 | |
| | (F241A) | |
| 10 | Amino acid | AEPKSCDKTHTCPPCPAPELLGGPSVFLFPPKPKDTLMISRTPEVTCVVVDVSHEDPEVKFN |
| | sequence of | WEVDGVEVHNAKTKPREEQYNSTERVVSVLTVLHQDWLNGEEEKCKVSNEALPAPIEKTISK |
| | human IgG1 Fc | TKGRPREPQVYTLPPSRDELTKNQVSLTCLVKGFEPSDIAVEWESNGQPENNYKTTPPVLDS |
| | polypeptide | DGSFFLYSKLTVDRSRWQQGNVITSCSVMHEALHNHYTQKSLSTSPGK |
| | comprising | |
| | mutations at | |
| | positions 317 | |
| | (K317E), 326 | |
| | (K326E), 339 | |
| | (A339T), 342 | |
| | (Q342R), and 414 | |
| | (K414R) | |
| 11 | Amino acid | AEPKSCDKTHTCPPCPAPELLGGPSVFLFPPKPKDTLMISRTPEVTCVVVDVSHEDPEVKFN |
| | sequence of | WEVDGVEVHNAKTKPREEQYNSTERVVSVLTVLHQDWLNGEEEKCKVSNEALPAPIEKTISK |
| | human IgG1 Fc | AKGQPREPQVYTLPPSRDELTKNQVSLTCLVKGFEPSDIAVEWESNGQPENNYKTTPPVLDS |
| | polypeptide | DGSFFLYSKLTVDRSRWQQGNVITSCSVMHEALHNHYTQKSLSTSPGK |
| | comprising | |
| | mutations at | |
| | positions 317 | |
| | (K317E), 326 | |
| | (K326E) and 414 | |
| | (K414R) | |
| 12 | Amino acid | AEPKSCDKTHTCPPCPAPELLGGPSVALFPPKPKDTLMISRTPEVTCVVVDVSHEDPEVKFN |
| | sequence of | WEVDGVEVHNAKTKPREEQYNSTERVVSVLTVLHQDWLNGEEEKCKVSNEAFPAPIEKTISK |
| | human IgG1 Fc | TKGRPREPQVYTLPPSRDELTKNQVSLTCLVKGFEPSDIAVEWESNGQPENNYKTTPPVLDS |
| | polypeptide | DGSFFLYSKLTVDRSRWQQGNVITSCSVMHEALHNHYTQKSLSTSPGK |
| | comprising | |
| | mutations at | |
| | positions 241 | |
| | (F241A), 317 | |
| | (K317E), 326 | |
| | (K326E), 328 | |
| | (L328F), 339 | |
| | (A339T), 342 | |
| | (Q342R) and 414 | |
| | (K414R) | |
| 13 | Amino acid | AEPKSCDKTHTCPPCPAPELLGGPSVALFPPKPKDTLMISRTPEVTCVVVDVSHEDPEVKFN |
| | sequence of | WEVDGVEVHNAKTKPREEQYNSTERVVSVLTVLHQDWLNGEEEKCKVSNEALPAPIEKTISK |
| | human IgG1 Fc | TKGRPREPQVYTLPPSRDELTKNQVSLTCLVKGFEPSDIAVEWESNGQPENNYKTTPPVLDS |
| | polypeptide | DGSFFLYSKLTVDRSRWQQGNVITSCSVMHEALHNHYTQKSLSTSPGK |
| | comprising | |
| | mutations at | |
| | positions 241 | |
| | (F241A), 317 | |
| | (K317E), 326 | |
| | (K326E), 339 | |
| | (A339T), 342 | |
| | (Q342R) and 414 | |
| | (K414R) | |

1. An Fc-containing polypeptide comprising one or more mutations at positions selected from the group consisting of: 223, 246, 250, 272, 284, 302, 305, 307, 309, 317, 320, 322, 326, 328 332, 335, 339, 340, 342, 360, 377, 391, 409, 412, 414, 421, 422, 437 and 439 of the Fc region of the Fc-containing polypeptide, wherein the numbering is according to the EU index as in Kabat, wherein the polypeptide comprises enhanced binding to human and mouse FcγRIIB when compared to a parent Fc-containing polypeptide.

2. The Fc-containing polypeptide of claim 1, comprising a mutation selected from the group consisting of:
a) T223I
b) K246R
c) K246E
d) T250A
e) E272K
f) V284A
g) V305A
h) V302A
i) T307A
j) L309S
k) K317E
l) N315S
m) K320R
n) K322E
o) K326E
p) L328M
q) I332T
r) T335A
s) A339T
t) K340E
u) Q342R
v) K360R
w) K360E
x) I377V
y) Y391H
z) K409R
aa) V412A
bb) K414R
cc) N421D
dd) V422D
ee) T437A
ff) K439E
gg) S440P.

3. The Fc-containing polypeptide of claim 1, comprising one or more mutations at positions selected from the group consisting of: 246, 307, 317, 326, 332, 339, 360, 409, 414 and 421.

4. The Fc-containing polypeptide of claim 3, comprising a mutation selected from the group consisting of:
a) K246R
b) K246E
c) T307A
d) K317E
e) K326E
f) A339T
g) K360R
h) K360E
i) K409R
j) K414R, and
k) N421D.

5. The Fc-containing polypeptide of claim 1, comprising one or more mutations at positions selected from the group consisting of: 317, 326, and 414.

6. The Fc-containing polypeptide of claim 5, comprising the mutations are selected from the group consisting of:
a) K317E
b) K326E, and
c) K414R.

7. The Fc polypeptide of claim 1, comprising mutations at amino acid positions 317, 326, and 414.

8. The Fc-containing polypeptide of claim 1, comprising one of the following sets of mutations:
a) K317E/K326E/K414R
b) K317E/K326E/A339T/Q342R/K414R
c) V284A/V305A/T307A/K360R
d) T250A/E272K/K360E/V422D/T437A
e) T223I/K320R/K322E
f) L309S/K340E/V412A
g) V302A/K326E/L328M/T335A/Y391H/K409R
h) K246R/A339T/K409R
i) K246E/T307A/K326E
j) N315S/I332T/N421D/S440P
k) K326E/N421D/K439E
l) K326E/I332T/I377V.

9. The Fc-containing polypeptide of claim 8, wherein the Fc-polypeptide comprises mutations K317E, K326E and K414R.

10. The Fc-containing polypeptide of claim 1, wherein the Fc-containing polypeptide further comprises one or more mutations at positions selected from the group consisting of: 241, 243, 252, 254, 256, 264, 267, 328, 339, 342, 433, and 434 of the Fc region, wherein the numbering is according to the EU index as in Kabat.

11. The Fc-containing polypeptide of claim 1, wherein the Fc-containing polypeptide comprises mutations at positions 241, 317, 326 and 414.

12. The Fc-containing polypeptide of claim 10, wherein the mutations are F241A, K317E, K326E and K414R.

13. (canceled)

14. (canceled)

15. (canceled)

16. The Fc-containing polypeptide of claim 1, wherein the Fc containing polypeptide is an antibody or an antibody fragment.

17. The Fc-containing polypeptide of claim 1, wherein the Fc-containing polypeptide is an IgG1 antibody or an antibody fragment.

18. The Fc polypeptide of claim 1, wherein the isolated polypeptide has one or more of the following properties when compared to a parent Fc-containing polypeptide:
a) reduced effector properties; and
b) increase anti-inflammatory property.

19. A method of producing an Fc-containing polypeptide in a host cell comprising:
a) providing a genetically modified host cell that has been genetically engineered to produce an Fc-containing polypeptide comprising sialylated N-glycans, wherein the host cell comprises a nucleic acid encoding an Fc-containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437, and 439 of the Fc region of the Fc-containing polypeptide, wherein the numbering is according to the EU index as in Kabat.
b) culturing the host cell under conditions which cause expression of the Fc-containing polypeptide; and
c) isolating the Fc-containing polypeptide from the host cell.

20. A method of increasing the anti-inflammatory properties of an Fc-containing polypeptide comprising introducing one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437 of the Fc region of the Fc-containing polypeptide, wherein the numbering is according to the EU index as in Kabat; and wherein the Fc-containing polypeptide has improved binding to human and mouse FcγRIIB and increased anti-inflammatory properties when compared to a parent Fc-containing polypeptide.

21. (canceled)

22. A method of treating a subject in need thereof comprising: administering to the subject a therapeutically effective amount of an Fc-containing polypeptide comprising one or more mutations at positions selected from the group consisting of: 223, 246, 250, 272, 284, 302, 305, 307, 309, 317, 320, 322, 326, 328, 332, 335, 339, 340, 342, 360, 377, 391, 409, 412, 414, 421, 422, 437, of the Fc region of the Fc-containing polypeptide, wherein the numbering is according to the EU index as in Kabat.

23. (canceled)

24. (canceled)

25. A pharmaceutical formulation comprising:
a) the Fc-containing polypeptide of claim 1, and
b) a pharmaceutically acceptable carrier.


SEQUENCE LISTING (13 sequences)

SEQ ID NO: 1 - protein, 222 aa, Homo Sapiens
TCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 232 aa, Homo Sapiens
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 449 aa, Artificial Sequence
  Antibody Sequence
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 4 - DNA, 9180 bp, Artificial Sequence
  Plasmid
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacggaattcacgatgagattcccatccatcttcactgctgttttgttcgctgcttcctctgctttggctgacattcaaatgactcagtccccatcttccttgtctgcttccgttggtgacagagttactatcacttgtaaggcttcccagaacgttggaactaacgttgtttggtatcagcagaagccaggtaaggctccaaaggctttgattcactccgcttcatacagatactccggtgttccatccagattctctggttctggttccggtactgactttactttgactatctcctcattgcagccagaggacttcgctacttactactgtcagcagtacaagacttacccatacactttcggtcagggtaccaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgtggtggtggtggttccggtggtggtggttctggtggtggtggttctgtcgaccaattctctaactctacttccgcttcctctactgacgttacttcctcctcctctatttctacttcctccggttccgttactattacttcctctgaggctccagaatctgacaacggtacttctactgctgctccaactgaaacttctactgaggctcctactactgctattccaactaacggaacttccacagaggctccaacaacagctatccctacaaacggtacatccactgaagctcctactgacactactacagaagctccaactactgctttgcctactaatggtacatcaacagaggctcctacagatacaacaactgaagctccaacaactggattgccaacaaacggtactacttctgctttcccaccaactacttccttgccaccatccaacactactactactccaccatacaacccatccactgactacactactgactacacagttgttactgagtacactacttactgtccagagccaactactttcacaacaaacggaaagacttacactgttactgagcctactactttgactatcactgactgtccatgtactatcgagaagccaactactacttccactacagagtatactgttgttacagaatacacaacatattgtcctgagccaacaacattcactactaatggaaaaacatacacagttacagaaccaactacattgacaattacagattgtccttgtacaattgagaagtccgaggctcctgaatcttctgttccagttactgaatccaagggtactactactaaagaaactggtgttactactaagcagactactgctaacccatccttgactgtttccactgttgttccagtttcttcctctgcttcttcccactccgttgttatcaactccaacggtgctaacgttgttgttcctggtgctttgggattggctggtgttgctatgttgttcttgtaatagggccggccatttaaatacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctggatccgcggccgcttacgcgccgttcttcgcttggtcttgtatctccttacactgtatcttcccatttgcgtttaggtggttatcaaaaactaaaaggaaaaatttcagatgtttatctctaaggttttttctttttacagtataacacgtgatgcgtcacgtggtactagattacgtaagttattttggtccggtgggtaagtgggtaagaatagaaagcatgaaggtttacaaaaacgcagtcacgaattattgctacttcgagcttggaaccaccccaaagattatattgtactgatgcactaccttctcgattttgctcctccaagaacctacgaaaaacatttcttgagccttttcaacctagactacacatcaagttatttaaggtatgttccgttaacatgtaagaaaaggagaggatagatcgtttatggggtacgtcgcctgattcaagcgtgaccattcgaagaataggccttcgaaagctgaataaagcaaatgtcagttgcgattggtatgctgacaaattagcataaaaagcaatagactttctaaccacctgtttttttccttttactttatttatattttgccaccgtactaacaagttcagacaaattaattaacaccatgtcagaagatcaaaaaagtgaaaattccgtaccttctaaggttaatatggtgaatcgcaccgatatactgactacgatcaagtcattgtcatggcttgacttgatgttgccatttactataattctctccataatcattgcagtaataatttctgtctatgtgccttcttcccgtcacacttttgacgctgaaggtcatcccaatctaatgggagtgtccattcctttgactgttggtatgattgtaatgatgattcccccgatctgcaaagtttcctgggagtctattcacaagtacttctacaggagctatataaggaagcaactagccctctcgttatttttgaattgggtcatcggtcctttgttgatgacagcattggcgtggatggcgctattcgattataaggaataccgtcaaggcattattatgatcggagtagctagatgcattgccatggtgctaatttggaatcagattgctggaggagacaatgatctctgcgtcgtgcttgttattacaaactcgcttttacagatggtattatatgcaccattgcagatattttactgttatgttatttctcatgaccacctgaatacttcaaatagggtattattcgaagaggttgcaaagtctgtcggagtttttctcggcataccactgggaattggcattatcatacgtttgggaagtcttaccatagctggtaaaagtaattatgaaaaatacattttgagatttatttctccatgggcaatgatcggatttcattacactttatttgttatttttattagtagaggttatcaatttatccacgaaattggttctgcaatattgtgctttgtcccattggtgctttacttctttattgcatggtttttgaccttcgcattaatgaggtacttatcaatatctaggagtgatacacaaagagaatgtagctgtgaccaagaactacttttaaagagggtctggggaagaaagtcttgtgaagctagcttttctattacgatgacgcaatgtttcactatggcttcaaataattttgaactatccctggcaattgctatttccttatatggtaacaatagcaagcaagcaatagctgcaacatttgggccgttgctagaagttccaattttattgattttggcaatagtcgcgagaatccttaaaccatattatatatggaacaatagaaattaattaacaggccccttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttaatttgcaagctgcggcctaaggcgcgccaggccataatggcccaaatgcaagaggacattagaaatgtgtttggtaagaacatgaagccggaggcatacaaacgattcacagatttgaaggaggaaaacaaactgcatccaccggaagtgccagcagccgtgtatgccaaccttgctctcaaaggcattcctacggatctgagtgggaaatatctgagattcacagacccactattggaacagtaccaaacctagtttggccgatccatgattatgtaatgcatatagtttttgtcgatgctcacccgtttcgagtctgtctcgtatcgtcttacgtataagttcaagcatgtttaccaggtctgttagaaactcctttgtgagggcaggacctattcgtctcggtcccgttgtttctaagagactgtacagccaagcgcagaatggtggcattaaccataagaggattctgatcggacttggtctattggctattggaaccaccctttacgggacaaccaaccctaccaagactcctattgcatttgtggaaccagccacggaaagagcgtttaaggacggagacgtctctgtgatttttgttctcggaggtccaggagctggaaaaggtacccaatgtgccaaactagtgagtaattacggatttgttcacctgtcagctggagacttgttacgtgcagaacagaagagggaggggtctaagtatggagagatgatttcccagtatatcagagatggactgatagtacctcaagaggtcaccattgcgctcttggagcaggccatgaaggaaaacttcgagaaagggaagacacggttcttgattgatggattccctcgtaagatggaccaggccaaaacttttgaggaaaaagtcgcaaagtccaaggtgacacttttctttgattgtcccgaatcagtgctccttgagagattacttaaaagaggacagacaagcggaagagaggatgataatgcggagagtatcaaaaaaagattcaaaacattcgtggaaacttcgatgcctgtggtggactatttcgggaagcaaggacgcgttttgaaggtatcttgtgaccaccctgtggatcaagtgtattcacaggttgtgtcggtgctaaaagagaaggggatctttgccgataacgagacggagaataaataaacattgtaataagatttagactgtgaatgttctatgtaatatttttcgagatactgtatctatctggtgtaccgtatcactctggacttgcaaactcattgattacttgtgcaatgggcaagaaggatagctctagaaagaagaagaaaaaggagccgcctgaagagctggatctttccgaggttgttccaacttttggttatgaggaatttcatgttgagcaagaggagaatccggtcgatcaagacgaacttgacggccataatggcctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - protein, 214 aa, Artificial Sequence
  Antibody sequence
DIQMTQSPSSLSASVGDRVTITCKASQNVGTNVVWYQQKPGKAPKALIHSASYRYSGVPSRFSGSGSGTDFTLTISSLQPEDFATYYCQQYKTYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 570 aa, Artificial Sequence
  Linker
MRFPSIFTAVLFAASSALADIQMTQSPSSLSASVGDRVTITCKASQNVGTNVVWYQQKPGKAPKALIHSASYRYSGVPSRFSGSGSGTDFTLTISSLQPEDFATYYCQQYKTYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSVDQFSNSTSASSTDVTSSSSISTSSGSVTITSSEAPESDNGTSTAAPTETSTEAPTTAIPTNGTSTEAPTTAIPTNGTSTEAPTDTTTEAPTTALPTNGTSTEAPTDTTTEAPTTGLPTNGTTSAFPPTTSLPPSNTTTTPPYNPSTDYTTDYTVVTEYTTYCPEPTTFTTNGKTYTVTEPTTLTITDCPCTIEKPTTTSTTEYTVVTEYTTYCPEPTTFTTNGKTYTVTEPTTLTITDCPCTIEKSEAPESSVPVTESKGTTTKETGVTTKQTTANPSLTVSTVVPVSSSASSHSVVINSNGANVVVPGALGLAGVAMLFL

SEQ ID NO: 7 - protein, 232 aa, Artificial Sequence
  Amino acid sequence of human IgG1 Fc polypeptide comprising mutations F243A and V264A
AEPKSCDKTHTCPPCPAPELLGGPSVFLAPPKPKDTLMISRTPEVTCVVADVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 232 aa, Artificial Sequence
  Amino acid sequence of human IgG1 Fc polypeptide comprising mutations 243 (F243A), 264 (V264A), 267 (S267E) and 328 (L238F)
AEPKSCDKTHTCPPCPAPELLGGPSVFLAPPKPKDTLMISRTPEVTCVVADVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 232 aa, Artificial Sequence
  Amino acid sequence of human IgG1 Fc polypeptide comprising a mutation at position 241 (F241A)
AEPKSCDKTHTCPPCPAPELLGGPSVALFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 233 aa, Artificial Sequence
  Amino acid sequence of human IgG1 Fc polypeptide comprising mutations at positions 317 (K317E), 326 (K326E), 339 (A339T), 342 (Q342R), and 414 (K414R)
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGEEYKCKVSNEALPAPIEKTISKTKGRPREPQVYTLPPSRDELTKNQVSLTCLVKGFYPSDIAVEWESNGQPENNYKTTPPVLDSDGSFFLYSKLTVDRSRWQQGNVFSCSVMHEALHNHYTQKSLSLSPGK

SEQ ID NO: 11 - protein, 233 aa, Artificial Sequence
  Amino acid sequence of human IgG1 Fc polypeptide comprising mutations at positions 317 (K317E), 326 (K326E) and 414 (K414R)
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGEEYKCKVSNEALPAPIEKTISKAKGQPREPQVYTLPPSRDELTKNQVSLTCLVKGFYPSDIAVEWESNGQPENNYKTTPPVLDSDGSFFLYSKLTVDRSRWQQGNVFSCSVMHEALHNHYTQKSLSLSPGK

SEQ ID NO: 12 - protein, 233 aa, Artificial Sequence
  Amino acid sequence of human IgG1 Fc polypeptide comprising mutations at positions 241 (F241A), 317 (K317E), 326 (K326E), 328 (L328F), 339 (A339T), 342 (Q342R) and 414 (K414R)
AEPKSCDKTHTCPPCPAPELLGGPSVALFPPKPKDTLMISRTPEVTCVVVDVSHEDPEVKFNWYVDGVEVHNAKTKPREEQYNSTYRVVSVLTVLHQDWLNGEEYKCKVSNEAFPAPIEKTISKTKGRPREPQVYTLPPSRDELTKNQVSLTCLVKGFYPSDIAVEWESNGQPENNYKTTPPVLDSDGSFFLYSKLTVDRSRWQQGNVFSCSVMHEALHNHYTQKSLSLSPGK

SEQ ID NO: 13 - protein, 233 aa, Artificial Sequence
  Amino acid sequence of human IgG1 Fc polypeptide comprising mutations at positions 241 (F241A),317 (K317E), 326 (K326E), 339 (A339T), 342 (Q342R) and 414 (K414R)
AEPKSCDKTHTCPPCPAPELLGGPSVALFPPKPKDTLMISRTPEVTCVVVDVSHEDPEVKFNWYVDGVEVHNAKTKPREEQYNSTYRVVSVLTVLHQDWLNGEEYKCKVSNEALPAPIEKTISKTKGRPREPQVYTLPPSRDELTKNQVSLTCLVKGFYPSDIAVEWESNGQPENNYKTTPPVLDSDGSFFLYSKLTVDRSRWQQGNVFSCSVMHEALHNHYTQKSLSLSPGK